(19) United States
(12) Patent Application Publication
(10) Pub. No.: US 2003/0219837 A1
(43) Pub. Date: Nov. 27, 2003

(54) Integrin ligand

(76) Inventors: Anne E. Cress, Tucson, AZ (US); Albert Edge, Newton, MA (US)

Correspondence Address: FISH & RICHARDSON PC, 225 FRANKLIN ST, BOSTON, MA, 02110, US

(21) Appl. No.: 10/382,808

(22) Filed: Mar. 6, 2003

Related U.S. Application Data
(60) Provisional application No. 60/365,370, filed on Mar. 18, 2002.

(51) Int. Cl. C12Q 1/70; G01N 33/53; G01N 33/567

(52) U.S. Cl. 435/7.2; 435/5

(57) ABSTRACT

The invention includes ligands (and methods for identifying the ligands) that bind specifically to a naturally occurring variant of a cell surface molecule, such as a naturally occurring variant of an integrin. The invention includes ligands that bind a naturally occurring variant of an alpha₆ integrin, called alpha₆p. The invention also includes methods of diagnosis and/or treatment using the ligands. Preferred ligands bind to the naturally occurring variant of the cell surface molecule with a higher affinity than to the unmodified cell surface molecule.

### RELATED APPLICATIONS

This application claims priority to U.S. provisional application 60/365,370, filed on Mar. 18, 2002, the contents of which are incorporated herein by reference.

### GOVERNMENT SUPPORT

The work described herein was carried out, at least in part, using funds from a federal grant, number NCI/NIH CA 56666. The government may, therefore, have certain rights in the invention.

### TECHNICAL FIELD

This invention relates to integrin ligands.

### BACKGROUND

Integrins on the surface of cells are receptors for components of the extracellular matrix (ECM). Binding of cell surface integrins to ECM organizes cells into tissues and coordinates their cellular functions. The ECM provides a route for cell migrations and activates signal-transduction pathways that induce cell growth, proliferation, and gene expression. Membrane-bound integrins mediate cell-ECM adhesion by binding directly to components of the cytoskeleton and the ECM.

Integrins are heterodimers of α and β subunits. Their ligand-binding site is composed of parts of both chains. At least 22 integrin heterodimers are known in mammals, composed of 17 types of α subunits and 8 types of β subunits. A single β chain can interact with multiple α chains, forming integrins that bind different ligands. For example, the widely expressed α₆β₁ integrin binds at least two different regions of laminin. This diversity of integrin heterodimers provides a mechanism of regulatory control for embryogenesis and tissue proliferation and differentiation.

Integrins typically exhibit relatively low affinities for their ligands (dissociation constants K_{D} between 10⁻⁶ and 10⁻⁸ mol/liter) compared with the high affinities (K_{D} values of 10⁻⁹ to 10⁻¹¹ mol/liter) of typical cell-surface hormone receptors. However, hundreds or thousands of integrin molecules can be present on a typical cell interacting with ECM proteins. The low affinity but large number of integrin molecules allows a cell the versatility to either remain firmly anchored to the matrix, or to quickly make and break specific contacts with the ECM during cell migration. Cells may express several types of integrins, allowing them to selectively regulate the activity of each type to thereby fine-tune their interaction with the ECM. Tumor tissues are characterized by loss of integrins, altering their interaction with ECM.

### SUMMARY OF THE INVENTION

The widely expressed alpha₆ integrin is a 140-kDa laminin receptor. Alpha₆p integrin is a 70-kDa, naturally occurring truncated form of the alpha₆ integrin. Integrin alpha₆p is believed to contain amino acid sequences corresponding to exons 13-25 of alpha₆, which correspond to the extracellular “stalk region,” the transmembrane sequence, and the cytoplasmic tail of the full-length alpha₆ integrin. The light chains of alpha₆ and alpha₆p are identical. The alpha₆p variant pairs with either beta₁ or beta₄ integrin subunits. The alpha₆p variant is found in human prostate (DU145H, LnCaP, PC3) and colon (SW480) cancer cell lines but not in normal prostate (PrEC), breast cancer (MCF-7), or lung cancer (H69) cell lines or a variant of a prostate carcinoma cell line (PC3-N). Alpha₆p can also be located on the leading edge of migrating endothial cells, and can function as a marker for migrating cells. After its biosynthesis, the alpha₆p variant is retained on the cell surface with a half-life three times longer than alpha₆.

The invention includes, inter alia, ligands (and methods for identifying the ligands) that bind specifically to a naturally occurring variant (e.g., a naturally occurring proteolytic product) of a cell surface molecule, e.g., a cell surface adhesion molecule or a cell surface receptor that responds to signals from extracellular ligands, e.g., a naturally occurring variant of an integrin, syndecan, selectin, or CAM, e.g., a naturally occurring variant of an alpha₆ integrin, e.g., alpha₆p. Also included are related methods of diagnosis and/or treatment using the ligands. The naturally occurring variant can be produced by cleavage of a cell surface molecule, e.g., cleavage by a protease, e.g., a urokinase. In preferred embodiments, a novel N-terminal sequence is exposed by the protease cleavage. In preferred embodiments, the cell surface molecule has a different tissue or cell specificity than the naturally occurring variant, e.g., the naturally occurring variant is present specifically in a disease cell, e.g., a cancer cell. A preferred ligand binds to the naturally occurring variant of the cell surface molecule, e.g., alpha₆p, with a higher affinity than to the un-modified, e.g., full-length, cell surface molecule, e.g., alpha₆. The term “un-modified” refers to the variation that causes differential length between the 70 kDa PAGE form of alpha₆p and alpha₆ integrin. A preferred ligand binds to the naturally occurring variant of the cell surface molecule, e.g., alpha₆p, but does not substantially bind to the full-length cell surface molecule, e.g., alpha₆ integrin (alpha₆).

Accordingly, in one aspect, the invention features a method of evaluating an agent, e.g., screening for a ligand that binds specifically to a naturally occurring variant (e.g., a naturally occurring proteolytic product) of a cell surface molecule, e.g., a cell surface adhesion molecule or a cell surface receptor that responds to signals from extracellular ligands, e.g., a naturally occurring variant of an integrin, syndecan, selectin, or CAM, e.g., a naturally occurring variant of an alpha₆ integrin, e.g., alpha₆p. The method includes determining whether a test agent specifically interacts with, e.g., binds to, the naturally occurring variant, e.g., alpha₆p. In one embodiment, the method includes: (a) providing a test compound; (b) contacting the test compound with the naturally occurring variant, e.g., alpha₆p or a fragment thereof, e.g., an N-terminal fragment thereof; (c) optionally contacting the test compound with the full-length cell surface molecule, e.g., alpha₆; and (d) selecting a compound that binds to the naturally occurring variant, e.g., alpha₆p, with a higher binding affinity than to the unmodified protein, e.g., selecting a compound that binds to the naturally occurring variant, e.g., alpha₆p, and preferably does not substantially bind to the full-length cell surface molecule, e.g., alpha₆. A compound that binds to the naturally occurring variant, e.g., alpha₆p, but preferably does not substantially bind to the full-length cell surface molecule, e.g., alpha₆, is identified as a ligand that binds specifically to a naturally occurring variant of a cell surface molecule. The test compound can be a compound, e.g., a protein, from a library of candidate compounds, e.g., from a phage display library, e.g., a phage display library described herein.

In a preferred embodiment, the naturally occurring variant is a proteolytic product of the full-length cell surface molecule. In preferred embodiments, the proteolytic product has a newly exposed N-terminus that differs from the N-terminus of the full-length (uncleaved) protein. In one embodiment, the naturally occurring variant is attached to the surface of a cell when it is contacted with the test compound. The cell can be a mammalian cell, such as a mouse or human cell. A mouse cell can be from a mouse cell line, such as a 291, O3C, or O3R cell line. A human cell can be from a prostate, colon, breast, or keratinocyte tissue or cell line. Exemplary human cell lines include DU145H (prostate carcinoma), HaCaT (normal immortalized keratinocyte), PC3-N (prostate tumor), MCF-7 (breast tumor), PC3-ATCC (prostate tumor), LnCap (prostate carcinoma), H69, SW480 (colon carcinoma), PrEC (normal prostate cells), and PC3 (prostate tumor).

In a preferred embodiment, the naturally occurring variant is alpha₆p.

In a preferred embodiment, the test compound is a polypeptide, e.g., a polypeptide that includes a large variable domain. In a preferred embodiment, the test compound is an antibody or a ligand-binding fragment thereof.

In a preferred embodiment, the test compound is a peptide, e.g., a cyclic peptide or a linear peptide.

In a preferred embodiment, the test compound binds to an epitope that includes the N-terminus of the naturally occurring variant, e.g., the N-terminus of alpha₆p. In one embodiment, the N-terminal region of the naturally occurring truncated integrin is not accessible to the test agent in the full-length, mature form of the integrin. The term “N-terminal region” refers to amino acids that are within 15 amino acids of the N-terminus of the truncated integrin.

In a preferred embodiment, the test compound binds to a novel conformational epitope that is present in the naturally occurring variant but absent in the unmodified (e.g., full-length) cell surface molecule.

In one aspect the invention features a method of screening for a ligand that has an ability to bind to a naturally occurring truncated form of an alpha₆ integrin. The screen can be performed by (a) providing a library of test ligands, e.g., a display library, such as a phage display library, (b) contacting members of the library with a naturally occurring form of an alpha₆ integrin, e.g., an alpha₆p integrin, and (c) identifying one or more members of the library that bind to the naturally occurring truncated form. In one embodiment, one or more of the identified members of the library can be contacted to the full-length, mature alpha₆ integrin, and then one or more members selected based on their preferential binding to the naturally occurring truncated alpha₆ integrin, e.g., alpha₆p.

In a preferred embodiment the test ligand is covalently linked to a bacteriophage coat protein.

In another aspect, the invention features a ligand that has a higher binding affinity for a naturally occurring variant of a cell surface molecule, e.g., alpha₆p, than to the unmodified (e.g., full-length) cell surface molecule, e.g., alpha₆. In a preferred embodiment, the binding affinity of the ligand for the naturally occurring variant is at least 10%, 20%, 30%, 40%, 50%, 60%, 70%, 80%, or higher than its binding affinity for the unmodified (e.g., full-length) cell surface molecule. More preferably, the ligand binds to the naturally occurring variant, e.g., alpha₆p, but does not substantially bind to the unmodified (e.g., full-length) cell surface molecule, e.g., alpha₆. The phrase “does not substantially bind” means that the binding affinity is less than 10%, 8%, 6%, 4%, or 2% of the binding affinity for the naturally occurring variant. The ligand can be, e.g., a protein, e.g., an antibody, e.g., a monoclonal antibody, recombinant IgG, or ligand binding fragment thereof (e.g., Fab′, Fab, F(ab′)₂, single domain antibodies (DABs), Fv, scFv (single chain Fv), and the like); or a peptide, e.g., a synthetic peptide or scaffold peptide.

In an embodiment, the ligand binds to an epitope that includes the N-terminus of the naturally occurring variant, e.g., the N-terminus of alpha₆p. In an embodiment, the naturally occurring variant is a proteolytic product of the full-length form.

In an embodiment, the ligand binds to a novel conformational epitope that is present in the naturally occurring variant but absent in the unmodified (e.g., full-length) cell surface molecule.

In an embodiment, the ligand consists of a plurality of polypeptide chains.

In an embodiment, the ligand consists of an immunoglobulin variable domain.

In an embodiment, the ligand is an antibody, e.g., a recombinant antibody, or an antigen-binding portion thereof.

In an embodiment, the ligand is monospecific, e.g., the ligand is a monoclonal antibody or antigen-binding portion thereof.

In an embodiment, the ligand is a human antibody.

In an embodiment, the ligand includes at least one, and preferably two, heavy (H) chain variable regions (abbreviated herein as VH), and at least one and preferably two light (L) chain variable regions (abbreviated herein as VL).

In one embodiment, the ligand impairs or prevents interaction between alpha₆p and UPAR. In another embodiment, the ligand impairs or prevents interaction between alpha₆p and a tetraspanin.

A preferred embodiment includes a tetramer of two heavy immunoglobulin chains and two light immunoglobulin chains, wherein the heavy and light immunoglobulin chains are inter-connected by, e.g., disulfide bonds.

In preferred embodiments, the ligand is humanized, e.g., the ligand contains human gene sequences, e.g., human immunoglobulin gene sequences in addition to other, e.g., recombinant, DNA sequences.

In a preferred embodiment, the ligand is a synthetic polypeptide or peptide, e.g., a cyclic peptide or linear peptide, e.g., of less than 25 amino acids.

In a preferred embodiment, the ligand, e.g., the antibody, is linked to an amino acid sequence, e.g., a peptide tag. Known peptides that can be used for fusion with the protein of the present invention include, for example, FLAG (Hopp et al. (1988) BioTechnology 6:1204-1210), a histidine tag, e.g., 6x His or 10x His, influenza agglutinin (HA), human c-myc fragment, VSV-GP fragment, p18HIV fragment, T7-tag, HSV-tag, E-tag, SV40T antigen fragment, lck tag. Other proteins that can be used for fusion with the ligand include, for example, GST (glutathione-S-transferase), HA (influenza agglutinin), immunoglobulin constant region, é-galactosidase, MBP (maltose binding protein).

In a preferred embodiment, the ligand, e.g., antibody, is conjugated to, linked to or associated with, a diagnostic agent. The diagnostic agent can be a detectable moiety, e.g., a radioactive, fluorescent or luminescent moiety, e.g., a radioactive ion.

In a preferred embodiment, the antibody is conjugated to, linked to, or associated with, a therapeutic agent. The therapeutic agent can be a cytotoxic or cytostatic agent, e.g., a cytotoxic T-cell; a complement protein; ricin; saponin; pseudomonas exotoxin; pokeweed antiviral protein; diphtheria toxin; vinblastine; 4-desacetylvinblastine; vincristine; leurosidine; vindesine; an anti-metabolite such as cytosine arabinoside, fluorouracil, methotrexate or aminopterin; anthracyclines, mitomycin C; a vinca alkaloid; demecolcine; etoposide; mithramycin; an anti-tumor alkylating agent such as chlorambucil or melphalan; a DNA synthesis inhibitor such as daunorubicin, doxorubicin, adriamycin and the like. Thus, the cytotoxic or cytostatic agent can act through, e.g., cell-mediated cytotoxicity, complement-mediated lysis, apoptosis, direct antibody-induced cell signaling, or indirect signaling by mimicking or altering signal transduction pathways. A preferred cytotoxic agent is specifically cytotoxic to a diseased cell, e.g., a cancer cell, e.g., a prostate or colon cancer cell.

In another aspect, the invention features a ligand that prevents interaction between alpha6 and a protease, e.g., between alpha6 and urokinase. has a higher binding affinity for a naturally occurring variant of a cell surface molecule, e.g., alpha₆p, than to the unmodified (e.g., full-length) cell surface molecule, e.g., alpha₆.

In another aspect, the invention features a method of treating a subject, e.g., a human. The method includes identifying a subject in need of a ligand, e.g., a protein ligand, that has a higher binding affinity for a naturally occurring variant of a cell surface molecule, e.g., alpha6p, than for the unmodified (e.g., full-length) cell surface molecule, e.g., alpha6, and administering the ligand to the subject. In a preferred embodiment, the binding affinity of the ligand for the naturally occurring variant is at least 10%, 20%, 30%, 40%, 50%, 60, 70%, 80%, or higher (i.e., better) than its binding affinity for the unmodified (e.g., full-length) cell surface molecule, or for example, the binding affinity (Kd) of the ligand for the naturally occurring variant is at least 4, 10, 20, 50, 100, 500, or 1000 less than its binding affinity (Kd) for the unmodified (e.g., full-length) cell surface molecule. More preferably, the ligand binds to the naturally occurring variant, e.g., alpha₆p, but does not substantially bind to the unmodified (e.g., full-length) cell surface molecule, e.g., alpha₆. The ligand can be, e.g., a protein, e.g., an antibody, e.g., a monoclonal antibody, recombinant IgG, or ligand binding fragment thereof (e.g., Fab′, Fab, F(ab′)₂, single domain antibodies (DABs), Fv, scFv (single chain Fv), and the like); or a peptide, e.g., a synthetic peptide or scaffold peptide. The ligand can be a humanized antibody. In a preferred embodiment, the antibody is linked to a peptide tag, e.g., a His- or myc-tag.

In a preferred embodiment, the subject is at risk for, or has, a proliferative disorder, e.g., a cancer, such as prostate, skin, breast, lung, kidney, pancreatic, or colon cancer. In one embodiment the cancer is a metastatic cancer. A metastatic cancer is one that has spread from one location to another location, e.g., from a organ or tissue of origin to another part, or other parts, of the body. The ligand can be used, for example, to target metastatic cells.

In a preferred embodiment, the subject is at risk for, or has, an epithelial disorder, e.g., epidermolysis bullosa.

In another embodiment the subject has or is at risk for a bleeding disorder, such as hemophilia, including hemophilia A, hemophilia B, and vascular hemophilia (von Willebrand's disease). Hemophilia A is caused by a deficiency of active clotting factor VIII, and the less common hemophilia B (Christmas disease) is caused by a lack of active clotting factor IX. Vascular hemophilia is a congenital disease caused a deficiency of a blood factor (von Willebrand's Factor) that promotes platelet adhesion.

In certain embodiments, a subject in need of one of the treatments described herein can experience bleeding episodes due to surgery, trauma, or other forms of tissue damage; coagulophathy, including coagulopathy in multi-transfused subjects; congenital or acquired coagulation or bleeding disorders, including decreased liver function (“liver disease”); defective platelet function or decreased platelet number; lacking or abnormal essential clotting “compounds” (e.g., platelets or von Willebrand factor protein); increased fibrinolysis; anticoagulant therapy or thrombolytic therapy; stem cell transplantation. In one series of embodiments, the bleedings occur in organs such as the brain, inner ear region, eyes, liver, lung, tumour tissue, gastrointestinal tract; in another series of embodiments, it is diffuse bleeding, such as in haemorrhagic gastritis and profuse uterine bleeding. In another series of embodiments, the bleeding episodes are bleeding in connection with surgery or trauma in subjects having acute haemarthroses (bleedings in joints), chronic haemophilic arthropathy, haematomas, (e.g., muscular, retroperitoneal, sublingual and retropharyngeal), bleedings in other tissue, haematuria (bleeding from the renal tract), cerebral haemorrhage, surgery (e.g., hepatectomy), dental extraction, and gastrointestinal bleedings (e.g., UGI bleeds). In one embodiment, the medicament is for treating bleeding episodes due to trauma, or surgery, or lowered count or activity of platelets in a subject. In one embodiment, treatment of the subject provides a method for reducing clotting time in a subject. In a preferred embodiment, the ligand modulates the level (i.e., amount) or activity of alpha6p integrin in the subject. For example, the ligand can increase alpha6p recycling.

In a preferred embodiment, the ligand binds to an epitope that includes the N-terminus of the naturally occurring variant, e.g., the N-terminus of alpha₆p.

In a preferred embodiment, the ligand binds to a novel conformational epitope that is present in the naturally occurring variant but absent in the un-modified (e.g., full-length) cell surface molecule.

In a preferred embodiment, the ligand is an antibody, e.g., a recombinant antibody or monoclonal antibody or alpha₆p-binding portion thereof.

In a preferred embodiment, the antibody reacts with the N-terminus of alpha₆p or with a novel conformational epitope that is present in the naturally occurring variant but absent in the un-modified (e.g., full-length) cell surface molecule.

In a preferred embodiment, the antibody is humanized.

In a preferred embodiment, the ligand, e.g., the antibody, is linked to a non-immunoglobulin amino acid sequence, e.g., a peptide tag, e.g., the antibody is a His-tagged or myc-tagged antibody.

In a preferred embodiment, the antibody is conjugated to, linked to or associated with, a diagnostic agent. The diagnostic agent can be a detectable moiety, e.g., a radioactive, fluorescent or luminescent moiety, e.g., a radioactive ion.

In a preferred embodiment, the antibody is conjugated to, linked to, or associated with a therapeutic agent. The therapeutic agent can be a cytotoxic or cytostatic agent, e.g., a cytotoxic T-cell; a complement protein; ricin; saponin; pseudomonas exotoxin; pokeweed antiviral protein; diphtheria toxin; vinblastine; 4-desacetylvinblastine; vincristine; leurosidine; vindesine; an anti-metabolite such as cytosine arabinoside, fluorouracil, methotrexate or aminopterin; anthracyclines, mitomycin C; a vinca alkaloid; demecolcine; etoposide; mithramycin; an anti-tumor alkylating agent such as chlorambucil or melphalan; a DNA synthesis inhibitor such as daunorubicin, doxorubicin, adriamycin and the like. Thus, the cytotoxic or cytostatic agent can act through, e.g., cell-mediated cytotoxicity, complement-mediated lysis, apoptosis, direct antibody-induced cell signaling, or indirect signaling by mimicking or altering signal transduction pathways. A preferred cytotoxic agent is specifically cytotoxic to a diseased cell, e.g., a cancer cell, e.g., a prostate or colon cancer cell. In another embodiment, the antibody is conjugated to, linked to, or associated with a protease inhibitor, e.g., a urokinase inhibitor.

In a preferred embodiment, the ligand, e.g., the antibody, modulates an activity of alpha₆p in the cell or tissue of the subject.

In another aspect, the invention features a method of identifying a ligand that binds specifically to a naturally occurring truncated form of an alpha6 integrin, e.g., an alpha6p integrin. The method can include the steps of (a) providing a test ligand, e.g., a ligand selected from a library of compounds or from a phage display library, (b) contacting the test ligand with the truncated alpha6 integrin, and (c) identifying the test ligand as a ligand that binds specifically to the truncated alpha6 integrin if the test ligand binds to the truncated integrin but does not substantially bind to a full-length form of the integrin.

In another aspect, the invention features a method of detecting alpha6p in a sample. The method can include the steps of contacting the sample with a ligand that has a higher binding affinity for alpha6p than alpha6 integrin, and determining that the ligand binds alpha6p.

In one aspect, the invention features a method of preventing or inhibiting the interaction of alpha6 with a binding partner, e.g., a urokinase plasminogen activator receptor (uPAR), urokinase or CD151.

In one embodiment, the method includes providing an agent that interacts with the binding partner. For example, the agent can bind to uPAR or CD151. Binding of the agent can cause steric interference, a conformational change, or another interaction that prevent interaction between the binding partner and alpha6. For example, the agent can bind to an epitope on uPAR or CD151 that interacts with alpha6. Similarly, an agent that binds to urokinase and prevents cleavage of alpha6 by urokinase can be used.

In a related aspect, the invention features agents that bind to alpha6 integrin that interfere with interaction with uPAR, urokinase, or CD151. In one embodiment, the agent contacts alpha6p in the stalk region. The stalk region extends from the N-terminal amino acid of alpha6p to residue 1014 of SEQ ID NO:1. The agent can therefore contact the alpha6p, for example, in a region between residues 516-1014, 570-1014, or 587-1014 of SEQ ID NO:1.

In another embodiment, the agent contacts the binding partner, e.g., uPAR, urokinase or a tetraspanin, e.g., CD9 CD81, or CD151.

In one embodiment, binding of the agent to the binding partner does not interfere with interactions between the binding partner and substrates other than alpha6p. For example, an agent that binds uPAR will disrupt uPAR binding to alpha6p, but will not affect uPAR binding to urokinase plasminogen activator (uPA, urokinase), alpha5beta1 complex, beta3 integrin, beta2 integrin CD11b/CD18 (Mac-1), alpha3 integrin, etc. An agent that binds the tetraspanin CD151 can disrupt CD151 binding to alpha6p, but does not affect CD151 binding to another molecule, e.g., phosphoinositide kinases, alpha3beta1 and alpha7beta1 complexes, etc. In one embodiment, the agent is a small molecule or an antibody.

In one aspect the invention features a method of inhibiting cleavage of a laminin-binding (e.g., mature, full-length) alpha6 integrin in a sample, e.g., in a tissue sample, or in vivo. The method can include treatment of the sample or a subject with a urokinase inhibitor. Exemplary urokinase inhibitors include an amino terminal fragment (ATF; residues 1-125) of urokinase (i.e., urokinase plasminogen activator, uPA) and amiloride. PAI-1 and PAI-2 are also urokinase inhibitors. The gene for PAI-1, and means for its recombinant expression, are disclosed in Loskutoff et al., U.S. Pat. No. 4,952,512. Recombinant and native human PAI-2 is disclosed in Stephens et al., U.S. Pat. No. 5,422,090. The most widely studied uPA inhibitors may be within the 4-substituted benzo[b]thiophene-2-carboxamidine class of inhibitors, of which B428 (4-iodo-benzo-[b]thiophene-2-carboxamidine) and B623 are members. Two competitive inhibitors of uPA are p-aminobenzamidine and alpha.-N-benzylsulfonyl-p-aminophenylalanine. Epigallo-cathecin-3 gallate (EGCG) is a polyphenol found in green tea and was reported to bind uPA and inhibit its activity (Jankun et al., Nature 387:561, 1997).

In another aspect, the invention features a method of evaluating a subject, e.g., determining if a subject is at risk for a disease or disorder, such as a proliferative disorder, epithelial disorder or bleeding disorder. The method includes detecting the absence or presence of a complex between a ligand (e.g., a ligand described herein) and a naturally occurring variant of a cell surface protein, e.g., a ligand-alpha₆p complex, in a cell or tissue of the subject, e.g., in a biopsy or blood sample of the subject, or in vivo, and correlating a positive interaction between the ligand and the variant with an increased risk for the disease or disorder. In some embodiments, the method includes administering to the subject a ligand that has a higher binding affinity for the naturally occurring variant than to the un-modified (e.g., full-length) cell surface protein, e.g., a ligand that has a higher binding affinity for alpha₆p than to alpha₆, e.g., a ligand that binds to alpha6p but does not substantially bind to alpha₆.

In a preferred embodiment, the ligand is a protein, e.g., an antibody, e.g., a monoclonal antibody, recombinant IgG, or ligand binding fragment thereof (e.g., Fab′, Fab, F(ab′).sub.2, single domain antibodies (DABs), Fv, scFv (single chain Fv), and the like); or a peptide, e.g., a synthetic peptide. A preferred ligand is a recombinant antibody or alpha₆p-binding fragment thereof.

In one embodiment, the subject has or is at risk for a cancer, e.g., a prostate or colon cancer, or a metastatic cancer. In another embodiment, the subject has or is a risk for an epithelial disorder, such as a blistering disorder, e.g., epidermolysis bullosa. In another embodiment, the subject is has or is at risk for a bleeding disorder, such as hemophilia.

In a preferred embodiment, the antibody reacts with the N-terminus of the naturally occurring variant, e.g., the N-terminus of alpha₆p.

In a preferred embodiment, the antibody reacts with a novel conformational epitope that is present in the naturally occurring variant but absent in the un-modified (e.g., full-length) cell surface molecule.

In a preferred embodiment, the ligand is an antibody, e.g., a recombinant antibody or monoclonal antibody or alpha₆p-binding portion thereof.

In a preferred embodiment, the antibody is humanized.

In a preferred embodiment, the ligand, e.g., the antibody, is linked to an amino acid sequence, e.g., a peptide tag, e.g., the antibody is a His-tagged or myc-tagged antibody.

In a preferred embodiment, the antibody is conjugated to, linked to or associated with, a diagnostic agent, e.g., an imaging agent. The diagnostic agent can be a detectable moiety, e.g., a radioactive, fluorescent or luminescent moiety, e.g., a radioactive ion.

Detecting the absence or presence of the complex, e.g., the ligand-alpha6p complex, can be performed in vivo (e.g., using in vivo imaging techniques), or ex vivo (e.g., by detecting the absence or presence of a ligand-alpha6p complex in a biopsy tissue sample).

In a related aspect, the invention provides a method for evaluating a sample for a proliferative disorder or epithelial disorder, such as a cancer or a blistering disorder. The method can include detecting an N-terminally truncated alpha6 integrin variant in the cell or tissue using a ligand described herein, e.g., a ligand specific for the integrin variant. Detection of the variant can indicate a parameter of risk for a proliferative disorder, or if an epithelial tissue has an epithelial-specific disorder or risk for an epithelial disorder. The sample can be obtained from a mammal, such as a human or a mouse. The cell can be obtained from a tissue of the body, such as the prostate, breast, colon, lung, skin or other epithelial tissue. The method can also be used to monitor a treatment.

In another aspect, a method of evaluating a subject includes the steps of (a) providing or obtaining a biological sample of the of the subject, (b) testing for an interaction between a ligand and a naturally occurring truncated form of an integrin, e.g., an alpha6 integrin, and (c) correlating a positive interaction between the ligand and the naturally occurring truncated alpha6 integrin with an increased risk for a disease or disorder. In a preferred embodiment, the disorder is a proliferative disorder, e.g., a cancer, such as a skin, prostate, colon, breast, lung, kidney, or pancreatic cancer. In certain embodiments, the cancer is metastatic. In another preferred embodiment, the disorder is an epithelial disorder, such as an epidermolysis bullosa. In another preferred embodiment, the disorder is a bleeding disorder, such as hemophilia.

In a preferred embodiment, the biological sample is a tissue or fluid sample. Examples include biopsies or a blood, urine, on serum samples.

In another aspect, the invention features a kit, e.g., for diagnosis of a condition or disorder associated with a naturally occurring variant of a cell surface molecule, e.g., a condition or disorder associated with alpha₆p, e.g., cancer. The kit includes a ligand capable of detecting a naturally occurring variant of a cell surface molecule, e.g., a truncated form of alpha₆ integrin, e.g., alpha₆p integrin; and instructions for use to detect a naturally occurring variant of a cell surface molecule, e.g., a truncated form of alpha₆ integrin.

In a preferred embodiment, the ligand has a higher binding affinity for the naturally occurring variant of a cell surface molecule, e.g., alpha₆p, than for the un-modified, e.g., full-length, cell surface molecule, e.g., alpha₆. Preferably, the ligand binds to the naturally occurring variant but does not substantially bind to the un-modified, e.g., full-length, cell surface molecule.

The ligand can be, e.g., a protein, e.g., an antibody, e.g., a monoclonal antibody, recombinant IgG, or ligand binding fragment thereof (e.g., Fab′, Fab, F(ab′)₂, single domain antibodies (DABs), Fv, scFv (single chain Fv), and the like); or a peptide, e.g., a synthetic peptide or scaffold peptide.

In a preferred embodiment, the ligand binds to alpha₆p but does not substantially bind to alpha₆.

In another aspect, the invention features a nucleic acid, e.g., a recombinant nucleic acid, encoding a ligand, e.g., a synthetic protein ligand described herein.

In a preferred embodiment, the nucleic acid contains additional sequences suitable for expression of the ligand, e.g., the nucleic acid is a vector, e.g., a plasmid or viral vector.

In another aspect, the invention features a host cell, e.g., a mammalian cell, e.g., a human cell, that includes a nucleic acid that encodes a ligand described herein.

In another aspect, the invention features a method to identify a protease that cleaves an alpha6 integrin, e.g., to produce an N-terminally truncated alpha6 integrin, such as alpha₆p. The method can include providing a test preparation (e.g., a recombinant protein, or a sub-cellular fraction), contacting the test preparation with a full-length alpha6 integrin, and evaluating the contacted preparation for a truncated alpha6 integrin or for reduction in the amount of alpha6 integrin. The test preparation can be further fractionated to identify a moiety that cleaves the alpha6 integrin. The test preparation can be analyzed, e.g., using an immunoassay, mass spectroscopy, and protein sequencing, e.g., to identify a characteristic of the protease.

In another embodiment, a parameter (e.g., a profile) of a cell that produces alpha6p is compared to a parameter or profile of a cell that produces alpha6, but not alpha6p. For example, mRNA can be prepared from the respective cells and compared, e.g., using microarray analysis, subtractive hybridization, and/or differential display. mRNA sequences that are preferentially expressed in one cell but not the other can be evaluated. Such sequences can also be filtered to identify sequences that are related by sequence content (e.g., using BLAST) to a protease, a protease inhibitor, or a protease activator. Such sequences can be mediators that affect alpha6 production.

In another aspect, the invention features a method of identifying a ligand that prevents alpha6 cleavage. The method includes: contacting alpha6 with a test ligand to form a complex, contacting the complex with a protease, and evaluating cleavage of alpha6. A test ligand that reduces the rate or other parameter related to cleavage is thereby identified as a ligand that prevents alpha6 cleavage. The test ligand can be a test ligand described herein, e.g., a display library member. In a related aspect, the method includes contact a cell that produces alpha6p with a test ligand, maintaining the cell under conditions that allow interaction between the test ligand and the cell (e.g., internalization of the test ligand, or binding to the cell surface), and evaluating the cell for production of alpha6p. A test ligand that reduces the abundance or presences of alpha6p is thereby identified as a ligand that prevents alpha6 cleavage.

In still another aspect, the invention features a method of identifying ligands that bind to the alpha6, but not alpha6p and that prevent cleavage of alpha6. The method can include screening a library of test ligands for members that bind to alpha6, but that do not substantially bind to alpha6p, and evaluating cleavage of alpha6 in vitro or in vivo in the presence of such members. For example, a test ligand that binds to the cleavage site of alpha6 can be used to prevent alpha6p production.

In one aspect, the invention features a protease-resistant laminin-binding alpha6 integrin. The laminin-binding alpha6 integrin can be a full-length mature form of the alpha6 integrin. A laminin-binding alpha6 integrin can be a full length alpha6 integrin, and can bind, for example, laminin-1 (e.g., in pancreatic tissue), laminin-5 (e.g., in preneoplastic lesions), and laminin-10 (e.g., in stromal tissues).

In one embodiment, the alpha6 integrin is resistant to urokinase. In another embodiment, the protease-resistant alpha6 integrin has a mutation at a protease cleavage site, e.g., a urokinase cleavage site, and the mutation, e.g., at least one deletion, insertion or substitution of an amino acid residue, abolishes the cleavage site. The mutation can occur between amino acid residues 516-597, 570-597, or 587-597 of SEQ ID NO:1. For example, the mutation can mutate a large hydrophobic amino acid to alanine, or a charged amino acid to alanine. Another aspect of the invention features a method of producing a protease-resistant laminin-binding (e.g., full-length) alpha6 integrin. The method includes providing a host cell that contains a nucleotide sequence encoding a mutant laminin-binding alpha6 integrin polypeptide. The mutant alpha6 integrin polypeptide can have a deletion, insertion or substitution of an amino acid between residues 516-597, 570-597, or 587-597 of SEQ ID NO:1, which causes resistance to a protease, e.g., a urokinase. To produce the protease-resistant alpha6 integrin, the host cell can be cultured under conditions that allow expression of the nucleotide sequence. A related method includes providing a mutated alpha6 integrin polypeptide or ectodomain thereof, contacting the mutated polypeptide with a urokinase, and evaluating the mutated polypeptide, e.g., by a cleavage event. In one embodiment, the mutated polypeptide is expressed on the surface of a cell, e.g., using a heterologous nucleic acid introduced into the cell.

In another aspect, the invention includes a method that includes: providing a laminin-binding alpha6 integrin, and contacting the integrin with urokinase under conditions that cause cleavage of the alpha6 integrin. The method can be an in vitro or an in vivo method. For example, the method can include providing a cell in vitro that has a cell surface alpha6 integrin, and contacting the surface integrin with the urokinase. The method can further include evaluating binding to an extracellular matrix, e.g., a laminin-containing substrate. The method can further include evaluating the alpha6 integrin, e.g., for cleavage.

Another aspect of the invention features a method of treating a subject, e.g., a human or animal. The method includes identifying a subject in need of an agent that inhibits cleavage of a laminin-binding alpha6 integrin (e.g., a full-length alpha6 integrin), and administering such agent to the subject. The agent can be, for example, a urokinase inhibitor, such as a urokinase ATF, amiloride, PAI-1, PAI-2, B428, B623, p-aminobenzamidine, epigallo-cathecin-3 gallate, or alpha-N-benzylsulfonyl-p-aminophenylalanine. The subject can be treated with a combination of these agents.

In another aspect, the agent interacts with uPAR and consequently inhibits the ability of uPAR to interact with alpha6.

In another aspect, the agent interacts with the laminin-binding alpha6 integrin, and consequently inhibits the interaction of the laminin-binding alpha6 integrin with uPAR.

In another aspect the agent interacts with a tetraspanin, e.g., CD151, and consequently inhibits interaction of the tetraspanin with alpha6p.

One aspect of the invention features a method of treating a subject, when the subject is in need of an increased activity of a truncated alpha6 integrin, e.g., an alpha6p integrin. The method includes administering urokinase or an activator of urokinase, e.g., a growth factor or hormone. In one embodiment, the subject has a thrombotic disorder, e.g., an embolism.

In certain embodiments the growth factor can be platelet-derived growth factor (PDGF), basic fibroblast growth factor (bFGF), interleukin-2 (IL-2), vascular endothelial growth factor A (VEGF-A), epidermal growth factor (EGF), insulin-like growth factor-I (IGF-I), heregulin-beta, fibroblast growth factor (FGF)-2, hepatocyte growth factor/scatter factor (HGF/SF).

In another aspect, the invention features a method that includes providing a nucleic acid to a cell in a subject (e.g., using a viral vector or liposome), the nucleic acid comprising a sequence encoding a alpha6 integrin and an operably linked promoter. The nucleic acid can be targeted to cells, e.g., to migratory cells, or cells that have alpha6p on their surface. For example, the virus or liposome can include a ligand described herein attached to its surface to target. The vector can be used to increase levels of alpha6 integrin expression. In one embodiment, the alpha6 integrin is wildtype; in another embodiment, it is a mutated urokinase-resistant alpha6 integrin.

Alpha6p integrin is a naturally occurring truncated form of the alpha6 integrin. Alpha6p migrates as a 70 kDa protein on an SDS-PAGE gel (SDS-polyacrylamide gel electrophoresis). Integrin alpha6p is believed to contain amino acid sequences corresponding to the C-terminal region of alpha6, which corresponds to the region containing the extracellular “stalk,” the transmembrane sequence, and the cytoplasmic tail of the full-length alpha6 integrin. The compositions and methods of the invention provide isolated N-terminally truncated alpha6 integrin polypeptides, also referred to herein as “alpha6 integrin variants,” and methods of making and using the isolated polypeptides.

The invention includes, inter alia, an isolated polypeptide that includes a truncated alpha6 integrin. A “truncated alpha6 integrin,” also referenced herein as an “alpha6 integrin variant” or “truncated alpha6 polypeptide,” is an alpha6 integrin that does not include an amino acid sequence from the N-terminus of the alpha6 integrin. For example, a sequence corresponding to at least amino acids 1-516 of SEQ ID NO:1 is not included in the truncated alpha6 integrin. In other words, the amino acids encoded by exons 1-11 of the alpha6 cDNA (SEQ ID NO:2; GenBank Accession No. AH008066.1; see Table 1) are not present in the truncated alpha6 integrin. The sequence of SEQ ID NO:2 is as follows.

ATGGCCGCCG CCGGCCAGCT GTCCTTGCTC TACCTGTCGG CGGGGCTCCT GTCCCGGCTC
GGCGCAGCCT TCAACTTGGA CACTCGGGAG GACAACGTGA TCCGGAAATA TGGAGACCCC
GGGAGCCTCT TCGGCTTCTC GCTGGCCATG CACTGGCAAC TGCAGCCCGA GGACAAGCGG
CTGTTGCTCG TGGGGGCCCC GCGGGCAGAA GCGCTTCCAC TGCAGAGAGC CAACAGAACG
GGAGGGCTGT ACAGCTGCGA CATCACCGCC CGGGGGCCAT GCACGCGGAT CGAGTTTGAT
AACGATGCTG ACCCCACGTC AGAAAGCAAG GAAGATCAGT GGATGGGGGT CACCGTCCAG
AGCCAAGGTC CAGGGGGCAA GGTCGTGACA TGTGCTCACC GATATGAAAA AAGGCAGCAT
GTTAATACGA AGCAGGAATC CCGAGACATC TTTGCGCGGT GTTATGTCCT GAGTCAGAAT
CTCAGGATTG AAGACGATAT GGATGGGGGA GATTGGAGCT TTTGTGATGG GCGATTGAGA
GGCCATGAGA AATTTGGCTC TTGCCAGCAA GGTGTAGCAG CTACTTTTAC TAAAGACTTT
CATTACATTG TATTTGGAGC CCCGGGTACT TATAACTGGA AAGGGATTGT TCGTGTAGAG
CAAAAGAATA ACACTTTTTT TGACATGAAC ATCTTTGAAG ATGGGCCTTA TGAAGTTCGT
GGAGAGACTG AGCATGATGA AAGTCTCGTT CCTGTTCCTG CTAACAGTTA CTTAGGTTTT
TCTTTGGACT CAGGGAAAGG TATTCTTTCT AAAGATGAGA TCACTTTTGT ATCTGGTGCT
CCCAGACCCA ATCACAGTGG AGCCGTGGTT TTGCTCAAGA GAGACATGAA GTCTGCACAT
CTCCTCCCTG AGCACATATT CGATGGAGAA GGTCTGGCCT CTTCATTTGG CTATGATCTG
GCGGTGGTGG ACCTCAACAA GGATGGGTGG CAAGATATAG TTATTGGAGC CCCACAGTAT
TTTGATAGAG ATGGAGAAGT TGCAGGTGCA GTGTATGTCT ACATGAACCA GCAAGGCAGA
TGGAATAATG TGAAGCCAAT TCGTCTTAAT GGAACCAAAG ATTCTATGTT TGGCATTGCA
GTAAAAAATA TTGGAGATAT TAATCAAGAT GGCTACCCAG ATATTGCAGT TGGAGCTCCG
TATGATGACT TGGGAAAGGT TTTTATCTAT CATGGATCTG CAAATGGAAT AAATACCAAA
CCAACACAGG TTCTCAAGGG TATATCACCT TATTTTGGAT ATTCAATTGC TGGAAACATG
GACCTTGATC GAAATTCCTA CCCTGATGTT GCTGTTGGTT CCCTCTCAGA TTCAGTAACT
ATTTTCAGAT CCCGGCCTGT GATTAATATT CAGAAAACCA TCACAGTAAC TCCTAACAGA
ATTGACCTCC GCCAGAAAAC AGCGTGTGGG CCGCCTAGTG GGATATGCCT CCAGCTTAAA
TCCTGTTTTG AATATACTGC TAACCCCGCT GGTTATAATC CTTCAATATC AATTGTGGGC
ACACTTGAAG CTGAAAAAGA AAGAAGAAAA TCTGGCCTAT CCTCAAGAGT TCAGTTTCGA
AACCAAGGTT CTGAGCCCAA ATATACTCAA GAACTAACTC TGAAGAGGCA GAAACAGAAA
GTGTGCATGG AGGAAACCCT GTGGCTACAG GATAATATCA GAGATAAACT GCGTCCCATT
CCCATAACTG CCTCAGTGGA GATCCAAGAG CCAAGCTCTC GTAGGCGAGT GAATTCACTT
CCAGAAGTTC TTCCAATTCT GAATTCAGAT GAACCCAAGA CAGCTCATAT TGATGTTCAC
TTCTTAAAAG AGGGATGTGG AGACGACAAT GTATGTAACA GCAACCTTAA ACTAGAATAT
AAATTTTGCA CCCGAGAAGG AAATCAAGAC AAATTTTCTT ATTTACCAAT TCAAAAAGGT
GTACCAGAAC TAGTTCTAAA AGATCAGAAG GATATTGCTT TAGAAAThAC AGTGACAAAC
AGCCCTTCCA ACCCAAGGAA TCCCACAAAA GATGGCGATG ACGCCCATGA GGCTAAACTG
ATTGCAACGT TTCCAGACAC TTTAACCTAT TCTGCATATA GAGAACTGAG GGCTTTCCCT
GAGAAACAGT TGAGTTGTGT TGCCAACCAG AATGGCTCGC AAGCTGACTG TGAGCTCGGA
AATCCTTTTA AAAGAAATTC AAATGTCACT TTTTATTTGG TTTTAAGTAC AACTGAAGTC
ACCTTTGACA CCCCATATCT GGATATTAAT CTGAAGTTAG AAACAACAAG CAATCAAGAT
AATTTGGCTC CAATTACAGC TAAAGCAAAA GTGGTTATTG AACTGCTTTT ATCGGTCTCG
GGAGTTGCTA AACCTTCCCA GGTGTATTTT GGAGGTACAG TTGTTGGCGA GCAAGCTATG
AAATCTGAAG ATGAAGTGGG AAGTTTAATA GAGTATGAAT TCAGGGTAAT AAACTTAGGT
AAACCTCTTA CAAACCTCGG CACAGCAACC TTGAACATTC AGTGGCCAAA AGAAATTAGC
AATGGGAAAT GGTTGCTTTA TTTGGTGAAA GTAGAATCCA AAGGATTCGA AAAGGTAACT
TGTGAGCCAC AAAAGGAGAT AAACTCCCTG AACCTAACGG AGTCTCACAA CTCAAGAAAG
AAACGGGAAA TTACTGAAAA ACAGATAGAT GATAACAGAA AATTTTCTTT ATTTGCTGAA
AGAAAATACC AGACTCTTAA CTGTAGCGTG AACGTGAACT GTGTGAACAT CAGATGCCCG
CTGCGGGGGC TGGACAGCAA GGCGTCTCTT ATTTTGCGCT CGAGGTTATG GAACAGCACA
TTTCTAGAGG AATATTCCAA ACTGAACTAC TTGGACATTC TCATGCGAGC CTTCATTGAT
GTGACTGCTG CTGCCGAAAA TATCAGGCTG CCAAATGCAG GCACTCAGGT TCGAGTGACT
GTGTTTCCCT CAAAGACTGT AGCTCAGTAT TCGGGAGTAC CTTGGTGGAT CATCCTAGTG
GCTATTCTCG CTGGGATCTT GATGCTTGCT TTATTAGTGT TTATACTATG GAAGTGTGGT
TTCTTCAAGA GAAATAAGAA AGATCATTAT GATGCCACAT ATCACAAGGC TGAGATCCAT
GCTCAGCCAT CTGATAAAGA GAGGCTTACT TCTGATGCAT AG

TABLE 1
Exon boundaries as defined by nucleotide numbers in
SEQ ID NO:2 and amino acid residues in SEQ ID NO:1.

| | Nucleotides in | Amino Acids in |
|---|---|---|
| Exon | SEQ ID NO:2 | SEQ ID NO:1 |
| 1 | <1-182 | 1-60 |
| 2 | 183-307 | 61-102 |
| 3 | 308-387 | 103-128 |
| 4 | 388-643 | 129-214 |
| 5 | 644-775 | 215-258 |
| 6 | 776-986 | 259-329 |
| 7 | 987-1180 | 330-393 |
| 8 | 1181-1269 | 394-423 |
| 9 | 1270-1388 | 424-463 |
| 10 | 1389-1487 | 464-496 |
| 11 | 1488-1549 | 497-516 |
| 12 | 1550-1710 | 517-570 |
| 13 | 1711-1854 | 571-618 |
| 14 | 1855-1970 | 619-656 |
| 15 | 1971-2160 | 657-719 |
| 16 | 2161-2244 | 720-747 |
| 17 | 2245-2324 | 748-774 |
| 18 | 2325-2402 | 775-799 |
| 19 | 2403-2505 | 800-832 |
| 20 | 2506-2679 | 833-889 |
| 21 | 2680-2778 | 890-922 |
| 22 | 2779-2889 | 923-959 |
| 23 | 2890-2988 | 960-992 |
| 24 | 2989-3114 | 993-1034 |
| 25 | 2115->3222 | 1035-1073 |

SEQ ID NO:1 represents the amino acid sequence of an exemplary full-length alpha6 grin protein. SEQ ID NO:1 is as follows:

(SEQ ID NO:1)
MAAAGQLCLL YLSAGLLSRL GAAFNLDTRE DNVIRKYGDP GSLFGFSLAM HWQLQPEDKR
LLLVGAPRGE ALPLQRANRT GGLYSCDITA RGPCTRIEFD NDADPTSESK EDQWMGVTVQ
SQGPGGKVVT CAHRYEKRQH VNTKQESRDT FGRCYVLSQN LRIEDDMDGG DWSFCDGRLR
GHEKFGSCQQ GVAATFTKDF HYIVFGAPGT YNWKGIVRVE QKNNTFFDMN IFEDGPYEVG
GETEHDESLV PVPANSYLGF SLDSGKGIVS KDEITFVSGA PRANHSGAVV LLKRDMKSAH
LLPEHIFDGE GLASSFGYDV AVVDLNKDGW QDIVIGAPQY FDRDGEVGGA VYVYMNQQGR
WNNVKPIRLN GTKDSMFGIA VKNIGDINQD GYPDIAVGAP YDDLGKVFIY HGSANGINTK
PTQVLKGISP YFGYSIAGNM DLDRNSYPDV AVGSLSDSVT IFRSRPVINI QKTITVTPNR
IDLRQKTACG APSGICLQVK SCFEYTANPA GYNPSISIVG TLEAEKERRK SGLSSRVQFR
NQGSEPKYTQ ELTLKRQKQK VCMEETLWLQ DNIRDKLRPI PITASVEIQE PSSRRRVNSL
PEVLPILNSD EPKTAHIDVH FLKEGCGDDN VCNSNLKLEY KFCTREGNQD KFSYLPIQKG
VPELVLKDQK DIALEITVTN SPSNPRNPTK DGDDAHEAKL IATFPDTLTY SAYRELRAFP
EKQLSCVANQ NGSQADCELG NPFKRNSNVT FYLVLSTTEV TFDTPYLDIN LKLETTSNQD
NLAPITAKAK VVIELLLSVS GVAKPSQVYF GGTVVGEQAM KSEDEVGSLI EYEFRVINLG
KPLTNLGTAT LNIQWPKEIS NGKWLLYLVK VESKGLEKVT CEPQKEINSL NLTESHNSRK
KREITEKQID DNRKFSLFAE RKYQTLNCSV NVNCVNIRCP LRGLDSKASL ILRSRLWNST
FLEEYSKLNY LDILMRAFID VTAAAENIRL PNAGTQVRVT VFPSKTVAQY SGVPWWIILV
AILAGILMLA LLVFILWKCG FFKRNKKDHY DATYHKAEIH AQPSDKERLT SDA

The N-terminal amino acid of the truncated alpha6 integrin can begin with an amino acid that corresponds to an amino acid between residues 516-597, 570-597, or 587-597 of SEQ ID NO:1, e.g., amino acid 587, 588, 589, 590, 591, 592, 593, 594, 595, or 596. In other words, the N-terminal amino acid of the truncated alpha6 integrin can begin with an amino acid encoded by a sequence of exons 12 or 13 of SEQ ID NO:2. The N-terminus of the truncated alpha6 integrin polypeptide can begin with the amino acid sequence arginine-valine-asparagine (RVN), RRVN (SEQ ID NO:4), RRRVN (SEQ ID NO:5), SRRRVN (SEQ ID NO:6), or SSRRRVN (SEQ ID NO:7). The N terminus of the truncated alpha6 integrin can include at least the amino acid sequence RVN of SEQ ID NO:1, but the truncated alpha6 integrin excludes amino acids 1-516 (encoded by exons 1-11 of alpha 6 integrin, which correspond to exons 1-11 (nucleotides 1-1856) of SEQ ID NO:2). In certain embodiments, the truncated alpha6 integrin includes only the extracellular region (the ectodomain), e.g., a sequence that terminates at an amino acid corresponding to an amino acid within ten amino acids of 1050 of SEQ ID NO:1.

A ligand of the invention can bind specifically to a truncated alpha6 integrin or to a C-terminal alpha6 integrin fragment that includes at least amino acids 596-1073 of the full-length alpha6 integrin (corresponding to amino acids 596-1073 of SEQ ID NO:1) and does not include a region corresponding to at least amino acids 1-516 of the full-length alpha6 integrin (e.g., amino acids 1-516 of SEQ ID NO:1). The C-terminal alpha6 integrin fragment can be located within a larger polypeptide sequence. For example, the fragment can be attached to a heterologous, non-integrin sequence, e.g., an N-terminal or C-terminal heterologous sequence, e.g., a tag or a domain of a protein and so forth. For example, the ectodomain of the truncated alpha6 integrin can be fused to a C-terminal tag, e.g., a hexahistidine sequence, an Fc domain, a GST protein, maltose binding protein, and so forth or to a reporter sequence, e.g., an enzyme such as alkaline phosphatase. In another example the ectodomain of the truncated alpha6 integrin is fused to a heterologous transmembrane sequence, a myristylation signal (e.g., the DAF signal), a viral protein, or a transmembrane sequence and a heterologous cytoplasmic domain.

The truncated alpha6 integrin can have the same N-terminus as a naturally-occurring truncated alpha6 integrin or the same sequence as a naturally-occurring truncated alpha6 integrin. Exemplary naturally-occurring truncated alpha6 integrins are found on cells of the prostate tumor cell lines DU145H (Rabinovitz et al., Clin. Exp. Metastasis 13:481-491, 1995), PC3 (Tran et al., Am. J. Pathol. 155:787-798), and LnCap; the colon cancer cell line SW480, and the normal immortalized keratinocyte cell line HaCat (Breitkrutz et al., Eur. J. Cell Biol. 75:273-286). Exemplary naturally-occurring truncated alpha6 integrins are also found on the leading edge of endothelial cells, thus colocalizing with the membrane-bound urokinase-type plasminogen activator receptor (uPAR). In addition, the truncated alpha6 integrins also colocalize on cells expressing PECAM-1 (platelet-endothelial cell adhesion molecule-1; also called CD31), which is associated with the regulation of cell migration and angiogenesis, e.g., an endothelial cell. A truncated alpha6 integrin can also localize to the membrane of metastatic cells and cells of invasive tumors.

The polypeptide can be a naturally occurring variant of the truncated alpha6 integrin, e.g., a variant that includes a sequence corresponding to a sequence within 517-1073, 571-1073, 588-1073, or 596-1073 of SEQ ID NO:1, e.g., substantially identical to amino acids 517-1073, 571-1073, 588-1073, or 596-1073 of SEQ ID NO:1. A sequence that is “substantially identical” is at least 85%, 90%, 95%, or 99% identical to an N-terminally truncated fragment of SEQ ID NO:1. For example, a sequence can differ by more than 5, 10, 20, 40, or 80 amino acids, but not more than 95 amino acids, from a sequence of the same length within SEQ ID NO:1, e.g., the sequence defined by amino acids 517-1073, 571-1073, 588-1073, or 596-1073 of SEQ ID NO:1. It is also possible for a sequence encoding a truncated alpha6p integrin to hybridize to a corresponding integrin sequence, e.g., a sequence that includes SEQ ID NO:2, under particular conditions, e.g., low, medium, high, or very high stringency conditions.

A sequence that “differs” from the specified sequence of SEQ ID NO:1, is a sequence in which an amino acid has been changed to a different amino acid, an amino acid has been deleted, or an amino acid has been added. In other embodiments, the truncated alpha6 polypeptide can be identical to an alpha6p amino acid sequence of a non-human animal, e.g., a non-human mammal (such as a, mouse, rat, pig, dog, or cat); a bird (such as a chicken); or an amphibian (such as a frog).

The truncated alpha6 integrin polypeptide can be a protease-resistant fragment of alpha6 integrin, and/or a naturally occurring fragment of alpha6 integrin. A “protease-resistant” polypeptide, as referenced herein, is as resistant or more resistant to degradation by proteases, such as kallikrein, plasmin, or urokinase, as a wildtype laminin-binding alpha6 integrin (e.g., a mature, full length integrin), e.g., as determined by an assay described herein. A polypeptide of the invention can be resistant to intracellular and extracellular proteases in cell culture in vivo, when the polypeptide is presented on the surface of a cell. By “naturally occurring” is meant a polypeptide that is found in vivo, in nature, in an unmodified biological system, such as a mammal. The truncated alpha6 integrin polypeptide can also be a polypeptide produced by cleavage of a laminin-binding integrin using urokinase. For example, the polypeptide can be produced recombinantly and then cleaved.

An “isolated” or “purified” polypeptide or protein is substantially free of cellular material or other contaminating proteins from the cell or tissue source from which the protein is derived, or substantially free from chemical precursors or other chemicals when chemically synthesized. An isolated alpha6 polypeptide can be associated with another protein, e.g., a beta integrin subunit. “Substantially free” means that a preparation of truncated alpha6 integrin protein is at least 10% pure. In a preferred embodiment, the preparation of truncated alpha6 integrin protein has less than about 30%, 20%, 10% and more preferably 5% (by dry weight), of non-truncated alpha6 integrin protein (also referred to herein as a “contaminating protein”), or of chemical precursors. When the truncated alpha6 integrin protein or biologically active portion thereof is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight. It is also possible for the isolated or purified polypeptide to be in a membrane preparation, liposome, or an organic solvent. In still another example, it is possible to produce the polypeptide in a heterologous cell, e.g., in a cell that does not naturally produce the truncated alpha6 integrin.

As used herein, “binding affinity” refers to the apparent association constant or Ka. The Ka is the reciprocal of the dissociation constant (Kd). A ligand may, for example, have a binding affinity of at least 10⁻⁵, 10⁻⁶, 10⁻⁷ or 10⁻⁸ M for a particular target molecule. Higher affinity binding of a ligand to a first target relative to a second target can be indicated by a higher Ka (or a smaller numerical value Kd) for binding the first target than the Ka (or numerical value Kd) for binding the second target. In such cases the ligand has specificity for the first target relative to the second target. A “higher binding affinity” refers to at least a 1.5 fold better binding affinity, e.g., 1.5 fold higher Ka, or 1.5 fold lower Kd. For example, binding affinities can be at least 2, 4, 5, 10, 20, 50, 100, 500, or 1000 fold better for one target (e.g., alpha6p) than another (e.g., alpha6).

Binding affinity can be determined by a variety of methods including equilibrium dialysis, equilibrium binding, gel filtration, ELISA, or spectroscopy (e.g., using a fluorescence assay). These techniques can be used to measure the concentration of bound and free ligand as a function of ligand (or target) concentration. The concentration of bound ligand ([Bound]) is related to the concentration of free ligand ([Free]) and the concentration of binding sites for the ligand on the target where (N) is the number of binding sites per target molecule by the following equation:

[Bound]=N·[Free]/((1/Ka)+[Free])

It is not always necessary to make an exact determination of Ka, though, since sometimes it is sufficient to obtain a quantitative measurement of affinity, e.g., determined using a method such as ELISA or FACS analysis, is proportional to Ka, and thus can be used for comparisons, such as determining whether a higher affinity is, e.g., 2 fold higher.

An “isolated composition” refers to a composition that is removed from at least 90% of at least one component of a natural sample from which the isolated composition can be obtained. Compositions produced artificially or naturally can be “compositions of at least” a certain degree of purity if the species or population of species of interests is at least 5, 10, 25, 50, 75, 80, 90, 95, 98, or 99% pure on a weight-weight basis.

An “epitope” refers to the site on a target compound that is bound by a ligand, e.g., a polypeptide ligand or an antigen-binding ligand (e.g., a Fab or antibody). In the case where the target compound is a protein, for example, an epitope may refer to the amino acids that are bound by the ligand.

As used herein, the term “substantially identical” (or “substantially homologous”) is used herein to refer to a first amino acid or nucleotide sequence that contains a sufficient number of identical or equivalent (e.g., with a similar side chain, e.g., conserved amino acid substitutions) amino acid residues or nucleotides to a second amino acid or nucleotide sequence such that the first and second amino acid or nucleotide sequences have similar activities. In the case of antibodies, the second antibody has the same specificity and has at least 50% of the affinity of the same.

Sequences similar or homologous (e.g., at least about 85% sequence identity) to the sequences disclosed herein are also part of this application. In some embodiment, the sequence identity can be about 85%, 90%, 91%, 92%, 93%, 94%, 95%, 96%, 97%, 98%, 99% or higher. Alternatively, substantial identity exists when the nucleic acid segments will hybridize under selective hybridization conditions (e.g., highly stringent hybridization conditions), to the complement of the strand. The nucleic acids may be present in whole cells, in a cell lysate, or in a partially purified or substantially pure form.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5.

As used herein, the term “homologous” is synonymous with “similarity” and means that a sequence of interest differs from a reference sequence by the presence of one or more amino acid substitutions (although modest amino acid insertions or deletions) may also be present. Presently preferred means of calculating degrees of homology or similarity to a reference sequence are through the use of BLAST algorithms (available from the National Center of Biotechnology Information (NCBI), National Institutes of Health, Bethesda Md.), in each case, using the algorithm default or recommended parameters for determining significance of calculated sequence relatedness. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the ligands of the invention may have mutations relative to a ligand described herein (e.g., a conservative or non-essential amino acid substitutions), which do not have a substantial effect on the polypeptide functions. Whether or not a particular substitution will be tolerated, i.e., will not adversely affect desired biological properties, such as binding activity can be determined as described in Bowie, et al. (1990) Science 247:1306-1310. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

A “non-essential” amino acid residue is a residue that can be altered from the wild-type sequence of the binding agent, e.g., the antibody, without abolishing or more preferably, without substantially altering a biological activity, whereas an “essential” amino acid residue results in such a change.

The terms “polypeptide” or “peptide” (which may be used interchangeably) refer to a polymer of three or more amino acids linked by a peptide bond, e.g., between 3 and 30, 12 and 60, or 30 and 300, or over 300 amino acids in length. The polypeptide may include one or more unnatural amino acids. Typically, the polypeptide includes only natural amino acids. A “protein” can include one or more polypeptide chains. Accordingly, the term “protein” encompasses polypeptides. A protein or polypeptide can also include one or more modifications, e.g., a glycosylation, amidation, phosphorylation, and so forth. The term “small peptide” can be used to describe a polypeptide that is between 3 and 30 amino acids in length, e.g., between 8 and 24 amino acids in length.

As used herein with respect to secreted or extracellular proteins, the term “truncated” refers to a mature molecule which lacks a region of a polymer chain present in another mature molecule, e.g., an N-terminal region. Accordingly, a secreted or extracellular molecule which is mature, i.e., which has its signal sequence removed, but otherwise full-length is not considered truncated.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, useful methods and materials are described below. The materials, methods, and examples are illustrative only and not intended to be limiting. Other features and advantages of the invention will be apparent from the following detailed description, and from the claims.

### DESCRIPTION OF DRAWINGS

FIG. 1 is a Western blot of alpha6 and alpha6p integrins immunoprecipitated from human cells. The DU145H cells were surface-biotinylated, and the alpha6 integrin was immunoprecipitated using either the GoH3, J1B5, AA6A, 4F10, or BQ16 antibodies, specific for human alpha6 integrin. The alpha5 integrin was precipitated from the lysate using the P1D6 antibody. The immunoprecipitations were analyzed using a 7.5% polyacrylamide gel under non-reducing conditions and the migration position of the biotinylated integrins are as indicated.

FIG. 2 is a Western blot of a two-dimensional polyacrylamide gel. Surface-biotinylated proteins from DU145H cells were immunoprecipitated using the GoH3 antibody and were analyzed first by 7.5% polyacrylamide gel electrophoresis under non-reducing conditions. The resulting lane was excised from the gel and placed on the top of a second 7.5% polyacrylamide gel. The positions of the migration of the integrins in the first gel are indicated at the top of the figure. Electrophoresis was then performed under reducing conditions. The resulting migration of the heavy chain (HC) and light chain (LC) and the molecular masses are indicated. The asterisk indicates a biotinylated protein band that was variably seen and is of unknown identity.

FIG. 3 is a pair of Western blots. FIG. 3A shows the results of coimmunoprecipitation experiments with A9, 439.9b, ASC3, or 3E1 antibodies, specific for human beta4 integrin, from surface-biotinylated HaCaT cells. FIG. 3B shows the results of coimmunoprecipitation experiments with a P4C10 antibody, specific for beta1 integrin, from surface-biotinylated DU145 cells and HaCaT cells. This Western blot also shows the results of a coimmunoprecipitation experiment with a J1B5 antibody, specific for alpha6 integrin, from the surface-biotinylated HaCaT cells. The immunoprecipitated proteins were analyzed using a 7.5% polyacrylamide gel under non-reducing conditions, and the migration positions of the biotinylated integrins are as indicated.

FIG. 4 is a panel of Western blots showing the results of immunoprecipitation experiments with GoH3, J1B5, and P4C10 antibodies. The alpha6- and the beta1-containing integrins were immunoprecipitated from the lysates of human DU145H, HaCaT, and H69 cells with either anti-alpha6 integrin antibodies GoH3 or J1B5, or an anti-beta1 integrin antibody P4C10. The precipitated proteins were analyzed on a 7.5% polyacrylamide gel under non-reducing conditions followed by Western blot (WB) analysis with the alpha6A-specific antibodies 4E9G8 or AA6A, which are specific for the cytoplasmic domain, or the anti-alpha6 integrin antibody A33, which is specific for the N terminus of alpha6. The migration position of a biotinylated integrin standard from DU145H cells are as indicated. The samples shown in the middle panel were electrophoresed on a separate gel, and the molecular mass of the alpha6A band is indicated relative to the adjacent panels by a solid bar. The asterisk indicates a biotinylated protein band that was variably seen and is of unknown identity.

FIG. 5 is a panel of Western blots showing the results of immunoprecipitation experiment with the GoH3 antibody. Alpha6-containing integrins were immunoprecipitated from the lysates of a normal, immortalized keratinocyte cell line (HaCaT) and a normal prostate epithelial cell line (PrEC), prostate cancer cell lines (PC3, PC3-N, and LnCaP), a breast cancer cell line (MCF-7), a colon carcinoma cell line (SW480), and a lung carcinoma cell line (H69). The immunoprecipitated proteins were analyzed on a 7.5% polyacrylamide gel under non-reducing conditions. The presence of alpha6p was detected by Western blot analysis using the AA6A antibody, specific for the human alpha6A light chain.

FIG. 6 shows the amino acid sequences of exons 1-25 of the alpha6 integrin (SEQ ID NO:1). MALDI mass spectrometry and HPLC coupled to mass spectrometry identified 10 noncontinuous amino acid fragments from the alpha6p variant (boxed sequences). These corresponded exactly to sequences contained within exons 13-25. Five of nine putative glycosylation sites are retained within exons 13-25 and are indicated in bold and underlined type. Ten of 20 cysteine residues (indicated by closed circles) are retained within exons 13-25.

FIG. 7 is a schematic comparing the alpha6 and alpha6p integrin proteins. Repeated domains (shaded rectangles) are indicated by Roman numerals I-VII. The putative ligand- and cation-binding domains are contained between repeated domains III and IV and domains V and VI, respectively. Exons 1-25 of the alpha6 integrin sequence are indicated. The mapped sequence positions of two anti-alpha6A antibodies (AA6A and 4E9G8) that recognize both alpha6 and the alpha6p variant are shown by an asterisk on the full-length alpha6 schematic. Conformationally dependent epitopes for anti-alpha6 integrin antibodies used for immunoprecipitation are not indicated on the schematic.

FIG. 8 is a Western blot (A) of alpha6 protein immunoprecipitated from surface-biotinylated DU145H cells, and a graph (B) of densitometry values illustrating the decay rates of alpha6, beta1 and alpha₆p. DU145H cells were surface-biotinylated and incubated for 24, 48, or 72 h, followed by lysis and immunoprecipitation with anti-alpha6 antibody GoH3. The samples were analyzed by a non-reducing 7.5% polyacrylamide gel, transferred to polyvinylidene fluoride membrane, reacted with peroxidase-conjugated streptavidin, and visualized by chemiluminescence (A). The asterisk indicates a biotinylated protein band that was variably seen and is of unknown identity. The film was digitized, and the densitometry values were analyzed for relative degradation rates of alpha6, beta1, and alpha₆p. Decay rates are illustrated in the graph in panel B.

FIG. 9 is a 1×TBE-1.5% agarose gel showing an RT-PCR amplification of the alpha6 integrin coding region and subsequent diagnostic digests. PCR primers that bracketed the integrin alpha6 coding region were used to amplify first strand cDNA generated from RNA isolated from DU145H cells (lane 1). To confirm the identity of the integrin alpha6 PCR product, aliquots were digested with four diagnostic restriction enzymes (EcoN I, lane 2; EcoR I, lane 3; Sma I, lane 4; and Xho I, lane 5), size separated, and visualized by ethidium bromide staining. The molecular mass standard is EcoR I/Hind III-digested DNA (lane M).

FIG. 10 is a Western blot (A) and graph (B) showing that calcium-induced normal keratinocyte differentiation increased alpha6p levels. The presence of alpha6 and alpha6p integrins was determined in normal 291 mouse keratinocytes, immortalized O3C nontumorigenic, and O3R tumorigenic derivatives. The cells were maintained in 0.4 mM calcium (low, lanes L) and switched to 0.14 mM (medium, lanes M) or 1.4 mM (high, lanes H) calcium medium at 60% confluency for 24 h of treatment, then frozen in a dry ice bath, and stored at 80° C. until analysis. Whole cell lysates (20 μg) were electrophoresed under non-reducing conditions on a 7.5% polyacrylamide gel and transferred to polyvinylidene fluoride membrane followed by Western blot analysis using anti-alpha6 integrin antibody, AA6A (A). The alpha6 and alpha6p integrin protein bands were scanned and quantitated using Scion Image and the results were graphed (B).

### DETAILED DESCRIPTION

The invention provides, in part, methods for identifying ligands that bind to naturally occurring variants of a cell surface molecule, e.g., naturally occurring variants of an integrin, e.g., alpha₆p. In many cases, the identified ligands are at least partially selective for the naturally occurring variant. The identified ligand may be, for example, a small peptide (e.g., a cyclic or linear peptide of between 7 and 25 amino acids), a polypeptide (e.g., a polypeptide of at least 20 amino acids), or a multi-chain protein (e.g., including at least two peptides or polypeptides). An example of a multi-chain protein is an antibody or an antigen binding fragment thereof

Alpha₆p is a truncated form of the alpha₆ integrin (Davis et al. (2001) Jour. Biol. Chem. 276:26099-26106). Alpha₆p integrin is believed to be derived through protease cleavage of alpha₆, exposing a novel N-terminus in alpha₆p. The amino acid sequence of human alpha₆ integrin is known (see Tamura et al. (1990) J. Cell Biol. 111:1593-1604; GenBank Accession No. NP_{—}000201) and is shown as SEQ ID NO:1.

The N-terminus of alpha₆p is contained within the amino acid sequence corresponding to exons 12-14 of alpha₆, e.g., within amino acid 517-656 of SEQ ID NO:1, preferably between amino acids 530-620 of SEQ ID NO:1, more preferably between amino acids 560-610 of SEQ ID NO:1, or at amino acid 596 (R) of SEQ ID NO:1. Without wanting to be bound by theory, it is believed that alpha₆p specific ligands can recognize, e.g., bind to, e.g., the N-terminus of alpha₆p, i.e., the N-terminal residues of alpha₆p which are not accessible to the ligand in the full-length length alpha₆ protein or to a novel conformational epitope formed by a novel secondary structure present in alpha₆p but absent in alpha₆. The ligand can bind to an epitope that includes, e.g., the N-terminus of alpha₆p, or at least one, two, or four amino acids that are within 5-100, 3-30, 1-20 residues from the N-terminus of alpha₆p. In one embodiment, the epitope includes the amino acid sequence RVN. The methods for identifying proteins that bind to alpha₆p include: providing a library and screening the library to identify a member that encodes a protein that binds to alpha₆p and does not substantially bind to alpha₆ integrin. The screening can be performed in a number of ways. For example, the library can be a display library as described herein.

The alpha₆p or a fragment thereof, e.g., a fragment corresponding to or including the N-terminus of alpha6p, can be recombinantly expressed and/or tagged. For example, the alpha6p can be expressed on the surface of a cell. The display library can be screened to identify members that specifically bind to the cell, e.g., only if the alpha6p is expressed. Alpha6p can also be purified and attached to a support, e.g., to paramagnetic beads or other magnetically responsive particle.

The laminin-binding form of the alpha6 integrin, including the full-length form of the integrin, interacts with the urokinase-type plasminogen activator receptor (uPAR) via the stalk region (see supra) of the integrin. Interaction with the receptor occurs in the cell membrane, and activation of the receptor, e.g., by urokinase, is believed to activate cleavage of the alpha6 integrin by urokinase (also called urokinase-type plasminogen activator; uPA), which creates the N-terminally truncated form, alpha6p integrin. Cleavage of alpha6 integrin causes cells to detach from the extracellular matrix (ECM), and more specifically, cleavage renders alpha6p incapable of interacting with laminin-5 and laminin-10; the full-length alpha6 integrin (as an alpha6beta4 complex) interacts with laminin-5 in preneoplastic lesions, and alpha6 integrin (as an alpha6beta1 complex) interacts with laminin-10 in stromal tissues. Alpha6p has been localized to the leading edge of endothelial cells, consistent with the localization pattern of uPAR. In addition, alpha6p has been localized to PECAM⁺ cells, metastatic cells, human prostate tumor cells, as well as cells of invasive tumors. These observations demonstrate that when cells receive a signal to migrate, urokinase may cleave alpha6 integrin to form alpha6p. Thus the presence of alpha6p on the surface of a cell can be indicative of migrating cells.

Urokinase is delivered in thrombolytic therapies to treat patients diagnosed with embolisms, such as pulmonary embolisms. Treatment with urokinase enhances the conversion of plasminogen to plasmin, the active fibrinolytic enzyme. Since the activity of urokinase can trigger cleavage of alpha6 integrin, and the subsequent formation of alpha6p integrin, activators of alpha6p integrin may also be used to treat clotting disorders, including thrombic disorders, e.g., embolisms.

Growth factors, such as epidermal growth factor (EGF), and hormones can trigger cleavage of the alpha6 integrin. Metastatic tissues may receive signals from autocrine or paracrine mechanisms. Inappropriate signaling, e.g. by growth factors, can lead to uncontrolled cell growth, and therefore lead to cancers, e.g., metastatic cancers and/or invasive tumors. Since alpha6p is associated with aberrant cell growth, it may have a role, active or passive in cancer growth. The methods of the invention function to disrupt the lateral interactions with other proteins, including tetraspanins, uPAR and urokinase.

The truncated integrin alpha6p interacts with the tetraspanin CD151 on migrating cells.

The colocalization of alpha6p with PECAM-1 supports the existence of a role for a truncated form of the integrin in cell migration, and suggests a role for such a polypeptide in angiogenesis. Angiogenic processes stimulate new blood vessel formation, which includes increased endothelial cell proliferation. Factors involved in angiogenesis, such as a truncated alpha6 integrin, e.g., alpha6p, may also potentiate angiogenesis-driven diseases such as diabetic retinopathy and certain tumors.

Factors involved in the process of angiogenesis, including a truncated form of alpha6 integrin, e.g., alpha6p, are candidate factors for angiogenesis-based therapies. For example, alph6p, or a fragment thereof, can be used to stimulate new blood vessel formation in the treatment of ischemic heart disease, for example, chronic myocardial ischemia or acute myocardial infarction (Ware and Simons, Nature Med. 3:158-164, 1997). Many patients with severe vascular disease that are not candidates for mechanical revascularization can benefit from angiogenesis-based therapy, including those patients with occlusion of vessels too small to be bypassed, those without conduits and those who are not surgical candidates because of concomitant disease. It has been calculated that 314 million disease cases in the U.S. and European Union may benefit from angiogenesis-based therapy (Miller and Abrams, Gen. Engin. News 18:1, 1998).

It is also useful to inhibit angiogenesis, e.g., in tumors. Inhibition can be effected by reducing cleavage of alpha6 integrin, e.g., by inhibiting urokinase activity, or by inhibiting interaction between alpha6 integrin and an alpha6 integrin interacting protein, e.g., urokinase, uPAR, or a tetraspanin.

Display Libraries

A display library is used to identify proteins that bind to the alpha₆p integrin but that do not substantially bind to the alpha₆ integrin. A display library can also be used to identify proteins that bind to full length or a laminin binding alpha6 integrin, and that also inhibit interaction between alpha6 and an alpha6 interacting protein, e.g., UPAR, a tetraspanin, or urokinase. Third, a display library can be used identify ligands that bind to an alpha6 interacting protein and that also inhibit interaction between alpha6 and the alpha6 interacting protein.

A display library is a collection of entities; each entity includes an accessible polypeptide component and a recoverable component that encodes or identifies the peptide component. The polypeptide component can be of any length, e.g. from three amino acids to over 300 amino acids. In a selection, the polypeptide component of each member of the library is probed with alpha₆p integrin or a fragment thereof (and optionally with alpha₆ integrin) and if the polypeptide component (a) binds to the alpha₆p integrin and (b) does not substantially bind to alpha₆ integrin, the display library member is identified, typically by retention on a support.

Retained display library members are recovered from the support and analyzed. The analysis can include amplification and a subsequent selection under similar or dissimilar conditions. For example, positive and negative selections can be alternated. The analysis can also include determining the amino acid sequence of the polypeptide component and purification of the polypeptide component for detailed characterization.

A variety of formats can be used for display libraries. Examples include the following.

Phage Display. One format utilizes viruses, particularly bacteriophages. This format is termed “phage display.” The polypeptide component is typically covalently linked to a bacteriophage coat protein. The linkage results form translation of a nucleic acid encoding the polypeptide component fused to the coat protein. The linkage can include a flexible peptide linker, a protease site, or an amino acid incorporated as a result of suppression of a stop codon. Phage display is described, for example, in Ladner et al., U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chem 274:18218-30, 1999; Hoogenboom et al. (1998) Immunotechnology 4:1-20; Hoogenboom et al. (2000) Immunol Today 2:371-8; Fuchs et al. (1991) BioTechnology 9:1370-1372; Hay et al. (1992) Hum Antibod Hybridomas 3:81-85; Huse et al. (1989) Science 246:1275-1281; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Rebar et al (1996) Methods Enzymol. 267:129-49; Hoogenboom et al. (1991) Nuc Acid Res 19:4133-4137; and Barbas et al. (1991) PNAS 88:7978-7982.

Phage display systems have been developed for filamentous phage (phage fl, fd, and M13) as well as other bacteriophage (e.g. T7 bacteriophage and lambdoid phages; see, e.g., Santini (1998) J. Mol. Biol. 282:125-135; Rosenberg et al. (1996) Innovations 6:1-6; Houshm et al. (1999) Anal Biochem 268:363-370). The filamentous phage display systems typically use fusions to a minor coat protein, such as gene III protein, and gene VIII protein, a major coat protein, but fusions to other coat proteins such as gene VI protein, gene VII protein, gene IX protein, or domains thereof can also been used (see, e.g., WO 00/71694). In a preferred embodiment, the fusion is to a domain of the gene III protein, e.g., the anchor domain or “stump,” (see, e.g., U.S. Pat. No. 5,658,727 for a description of the gene III protein anchor domain).

The valency of the polypeptide component can also be controlled. Cloning of the sequence encoding the polypeptide component into the complete phage genome results in multivariant display since all replicates of the gene III protein are fused to the polypeptide component. For reduced valency, a phagemid system can be utilized. In this system, the nucleic acid encoding the polypeptide component fused to gene III is provided on a plasmid, typically of length less than 700 nucleotides. The plasmid includes a phage origin of replication so that the plasmid is incorporated into bacteriophage particles when bacterial cells bearing the plasmid are infected with helper phage, e.g. M13K01. The helper phage provides an intact copy of gene III and other phage genes required for phage replication and assembly. The helper phage has a defective origin such that the helper phage genome is not efficiently incorporated into phage particles relative to the plasmid that has a wild type origin.

Bacteriophage displaying the polypeptide component can be grown and harvested using standard phage preparatory methods, e.g. PEG precipitation from growth media.

After selection of individual display phages, the nucleic acid encoding the selected polypeptide components, by infecting cells using the selected phages. Individual colonies or plaques can be picked, the nucleic acid isolated and sequenced.

Cell-based Display. In still another format the library is a cell-display library. Proteins are displayed on the surface of a cell, e.g., a eukaryotic or prokaryotic cell. Exemplary prokaryotic cells include E. coli cells, B. subtilis cells, spores (see, e.g., Lu et al. (1995) Biotechnology 13:366). Exemplary eukaryotic cells include yeast (e.g., Saccharomyces cerevisiae, Schizosaccharomyces pombe, Hanseula, or Pichia pastoris). Yeast surface display is described, e.g., in Boder and Wittrup (1997) Nat. Biotechnol. 15:553-557.

U.S. Provisional Patent Application No. Serial NO. 60/326,320, filed October 1, describes a yeast display system that can be used to display immunoglobulin proteins such as Fab fragments, and the use of mating to generate combinations of heavy and light chains..

In one embodiment, nucleic acid encoding immunoglobulin variable domains are cloned into a vector for yeast display. The cloning joins the nucleic acid encoding at least one of the variable domains with nucleic acid encoding a fragments of a yeast cell surface protein, e.g., Flo1, a-agglutinin, α-agglutinin, or fragments derived thereof e.g. Aga2p, Aga1p. A domain of these proteins can anchor the polypeptide encoded by the diversified nucleic acid sequence by a GPI-anchor (e.g. a-agglutinin, α-agglutinin, or fragments derived thereof e.g. Aga2p, Aga1p), by a transmembrane domain (e.g., Flo1). The vector can be configured to express two polypeptide chains on the cell surface such that one of the chains is linked to the yeast cell surface protein. For example, the two chains can be immunoglobulin chains.

Ribosome Display. RNA and the polypeptide encoded by the RNA can be physically associated by stabilizing ribosomes that are translating the RNA and have the nascent polypeptide still attached. Typically, high divalent Mg²⁺ concentrations and low temperature are used. See, e.g., Mattheakis et al. (1994) Proc. Natl. Acad. Sci. USA 91:9022 and Hanes et al. (2000) Nat Biotechnol. 18:1287-92; Hanes et al. (2000) Methods Enzymol. 328:404-30. and Schaffitzel et al. (1999) J Immunol Methods. 231(1-2):119-35.

Peptide-Nucleic Acid Fusions. Another format utilizes peptide-nucleic acid fusions. Polypeptide-nucleic acid fusions can be generated by the in vitro translation of mRNA that include a covalently attached puromycin group, e.g., as described in Roberts and Szostak (1997) Proc. Natl. Acad. Sci. USA 94:12297-12302, and U.S. Pat. No. 6,207,446. The mRNA can then be reverse transcribed into DNA and crosslinked to the polypeptide.

Other Display Formats. Yet another display format is a non-biological display in which the polypeptide component is attached to a non-nucleic acid tag that identifies the polypeptide. For example, the tag can be a chemical tag attached to a bead that displays the polypeptide or a radiofrequency tag (see, e.g., U.S. Pat. No. 5,874,214).

Scaffolds. Scaffolds for display can include: antibodies (e.g., Fab fragments, single chain Fv molecules (scFV), single domain antibodies, camelid antibodies, and camelized antibodies); T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains).

Appropriate criteria for evaluating a scaffolding domain can include: (1) amino acid sequence, (2) sequences of several homologous domains, (3) 3-dimensional structure, and/or (4) stability data over a range of pH, temperature, salinity, organic solvent, oxidant concentration. In one embodiment, the scaffolding domain is a small, stable protein domains, e.g., a protein of less than 100, 70, 50, 40 or 30 amino acids. The domain may include one or more disulfide bonds or may chelate a metal, e.g., zinc.

Examples of small scaffolding domains include: Kunitz domains (58 amino acids, 3 disulfide bonds), Cucurbida maxima trypsin inhibitor domains (31 amino acids, 3 disulfide bonds), domains related to guanylin (14 amino acids, 2 disulfide bonds), domains related to heat-stable enterotoxin IA from gram negative bacteria (18 amino acids, 3 disulfide bonds), EGF domains (50 amino acids, 3 disulfide bonds), kringle domains (60 amino acids, 3 disulfide bonds), fungal carbohydrate-binding domains (35 amino acids, 2 disulfide bonds), endothelin domains (18 amino acids, 2 disulfide bonds), and Streptococcal G IgG-binding domain (35 amino acids, no disulfide bonds).

Examples of small intracellular scaffolding domains include SH2, SH3, and EVH domains. Generally, any modular domain, intracellular or extracellular, can be used.

Another useful type of scaffolding domain is the immunoglobulin (Ig) domain. Methods using immunoglobulin domains for display are described below (see, e.g., “Antibody Display Libraries”).

Display technology can also be used to obtain ligands, e.g., antibody ligands, particular epitopes of a target. This can be done, for example, by using competing non-target molecules that lack the particular epitope or are mutated within the epitope, e.g., with alanine. Such non-target molecules can be used in a negative selection procedure as described below, as competing molecules when binding a display library to the target, or as a pre-elution agent, e.g., to capture in a wash solution dissociating display library members that are not specific to the target.

Synthetic Peptides. The targeting agent can include a synthetic peptide. A synthetic peptide is an artificial peptide of 30 amino acids or less. The synthetic peptide can include one or more disulfide bonds. Other synthetic peptides, so-called “linear peptides,” are devoid of cysteines. Synthetic peptides may have little or no structure in solution (e.g., unstructured), heterogeneous structures (e.g., alternative conformations or “loosely structured), or a singular native structure (e.g., cooperatively folded). Some synthetic peptides adopt a particular structure when bound to a target molecule. Some exemplary synthetic peptides are so-called “cyclic peptides” that have one disulfide bond, and a loop of about 4 to 12 non-cysteine residues.

Small synthetic peptides offer several advantages: First, the mass per binding site is low, e.g., such low molecular weight peptide domains can show higher bind activity per gram. Second, the possibility of non-specific binding and/or high antigenicity is minimal because there is only a small surface available. Third, small peptides can be engineered to have unique tethering sites such as terminal polylysine segments, e.g., by chemical synthetic methods. Fourth, small peptides can be combined into homo- or hetero-multimers to give either hybrid binding or avidity effects. Fifth, a constrained polypeptide structure is likely to retain its functionality in a variety of contexts.

Iterative Selection. In one preferred embodiment, display library technology is used in an iterative mode. A first display library is used to identify one or more ligands for a target. These identified ligands are then varied using a mutagenesis method to form a second display library. Higher affinity ligands are then selected from the second library, e.g., by using higher stringency or more competitive binding and washing conditions.

In some implementations, the mutagenesis is targeted to regions known or likely to be at the binding interface. If, for example, the identified ligands are antibodies, then mutagenesis can be directed to the CDR regions of the heavy or light chains as described herein. Further, mutagenesis can be directed to framework regions near or adjacent to the CDRs. In the case of antibodies, mutagenesis can also be limited to one or a few of the CDRs, e.g., to make precise step-wise improvements. Likewise, if the identified ligands are enzymes, mutagenesis can be directed to the active site and vicinity.

Some exemplary mutagenesis techniques include: error-prone PCR (Leung et al. (1989) Technique 1:11 - 15), recombination, DNA shuffling using random cleavage (Stemmer (1994) Nature 389-391; termed “nucleic acid shuffling”), RACHITT™ (Coco et al. (2001) Nature Biotech. 19:354), site-directed mutagenesis (Zooler et al. (1987) Nucl Acids Res 10:6487-6504), cassette mutagenesis (Reidhaar-Olson (1991) Methods Enzymol. 208:564-586) and incorporation of degenerate oligonucleotides (Griffiths et al. (1994) EMBO J 13:3245).

In one example of iterative selection, the methods described herein are used to first identify a protein ligand from a display library that binds a alpha₆p with at least a minimal binding specificity for a target or a minimal activity, e.g., an equilibrium dissociation constant for binding of greater than 1 nM, 10 nM, or 100 nM. The nucleic acid sequence encoding the initial identified protein ligand are used as a template nucleic acid for the introduction of variations, e.g., to identify a second protein ligand that has enhanced properties (e.g., binding affinity, kinetics, or stability) relative to the initial protein ligand.

Off-Rate Selection. Since a slow dissociation rate can be predictive of high affinity, particularly with respect to interactions between polypeptides and their targets, the methods described herein can be used to isolate ligands with a desired kinetic dissociation rate (i.e. reduced) for a binding interaction to a target.

To select for slow dissociating ligands from a display library, the library is contacted to an immobilized target. The immobilized target is then washed with a first solution that removes non-specifically or weakly bound biomolecules. Then the immobilized target is eluted with a second solution that includes a saturation amount of free target, i.e., replicates of the target that are not attached to the particle. The free target binds to biomolecules that dissociate from the target. Rebinding is effectively prevented by the saturating amount of free target relative to the much lower concentration of immobilized target.

The second solution can have solution conditions that are substantially physiological or that are stringent. Typically, the solution conditions of the second solution are identical to the solution conditions of the first solution. Fractions of the second solution are collected in temporal order to distinguish early from late fractions. Later fractions include biomolecules that dissociate at a slower rate from the target than biomolecules in the early fractions.

Further, it is also possible to recover display library members that remain bound to the target even after extended incubation. These can either be dissociated using chaotropic conditions or can be amplified while attached to the target. For example, phage bound to the target can be contacted to bacterial cells.

Selecting or Screening for Specificity. The display library screening methods described herein can include a selection or screening process that discards display library members that bind to a non-target molecule. An example of a non-target molecule is full-length alpha₆ integrin.

In one implementation, a so-called “negative selection” step is used to discriminate between the target and related non-target molecule and a related, but distinct non-target molecules. The display library or a pool thereof is contacted to the non-target molecule. Members of the sample that do not bind the non-target are collected and used in subsequent selections for binding to the target molecule or even for subsequent negative selections. The negative selection step can be prior to or after selecting library members that bind to the target molecule.

In another implementation, a screening step is used. After display library members are isolated for binding to the target molecule, each isolated library member is tested for its ability to bind to a non-target molecule (e.g., a non-target listed above). For example, a high-throughput ELISA screen can be used to obtain this data. The ELISA screen can also be used to obtain quantitative data for binding of each library member to the target. The non-target and target binding data are compared (e.g., using a computer and software) to identify library members that specifically bind to the target MHC-peptide complex.

Diversity

Display libraries include variation at one or more positions in the displayed polypeptide. The variation at a given position can be synthetic or natural. For some libraries, both synthetic and natural diversity are included.

Synthetic Diversity. Libraries can include regions of diverse nucleic acid sequence that originate from artificially synthesized sequences. Typically, these are formed from degenerate oligonucleotide populations that include a distribution of nucleotides at each given position. The inclusion of a given sequence is random with respect to the distribution. One example of a degenerate source of synthetic diversity is an oligonucleotide that includes NNN wherein N is any of the four nucleotides in equal proportion.

Synthetic diversity can also be more constrained, e.g., to limit the number of codons in a nucleic acid sequence at a given trinucleotide to a distribution that is smaller than NNN. For example, such a distribution can be constructed using less than four nucleotides at some positions of the codon. In addition, trinucleotide addition technology can be used to further constrain the distribution.

So-called “trinucleotide addition technology” is described, e.g., in U.S. Pat. No. 5,869,644. Oligonucleotides are synthesized on a solid phase support, one codon (i.e., trinucleotide) at a time. The support includes many functional groups for synthesis such that many oligonucleotides are synthesized in parallel. The support is first exposed to a solution containing a mixture of the set of codons for the first position. The unit is protected so additional units are not added. The solution containing the first mixture is washed away and the solid support is deprotected so a second mixture containing a set of codons for a second position can be added to the attached first unit. The process is iterated to sequentially assemble multiple codons. Trinucleotide addition technology enables the synthesis of a nucleic acid that at a given position can encoded a number of amino acids. The frequency of these amino acids can be regulated by the proportion of codons in the mixture. Further the choice of amino acids at the given position is not restricted to quadrants of the codon table as is the case if mixtures of single nucleotides are added during the synthesis.

Natural Diversity. Libraries can include regions of diverse nucleic acid sequence that originate (or are synthesized based on) from different naturally occurring sequences. An example of natural diversity that can be included in a display library is the sequence diversity present in immune cells (see also below). Nucleic acids are prepared from these immune cells and are manipulated into a format for polypeptide display. Another example of naturally-diversity is the diversity of sequences among different species of organisms. For example, diverse nucleic acid sequences can be amplified from environmental samples, such as soil, and used to construct a display library.

Antibody Display Libraries

In one embodiment, the display library presents a diverse pool of polypeptides, each of which includes an immunoglobulin domain, e.g., an immunoglobulin variable domain. Display libraries are particular useful, for example for identifying human or “humanized” antibodies that recognize human antigens. Such antibodies can be used as therapeutics to treat human disorders such as cancer. Since the constant and framework regions of the antibody are human, these therapeutic antibodies may avoid themselves being recognized and targeted as antigens. The constant regions are also optimized to recruit effector functions of the human immune system. The in vitro display selection process surmounts the inability of a normal human immune system to generate antibodies against self-antigens.

A typical antibody display library displays a polypeptide that includes a VH domain and a VL domain. An “immunoglobulin domain” refers to a domain from the variable or constant domain of immunoglobulin molecules. Immunoglobulin domains typically contain two β-sheets formed of about seven β-strands, and a conserved disulphide bond (see, e.g., A. F. Williams and A. N. Barclay 1988 Ann. Rev Immunol. 6:381-405). The display library can display the antibody as a Fab fragment (e.g., using two polypeptide chains) or a single chain Fv (e.g., using a single polypeptide chain). Other formats can also be used.

As in the case of the Fab and other formats, the displayed antibody can include a constant region as part of a light or heavy chain. In one embodiment, each chain includes one constant region, e.g., as in the case of a Fab. In other embodiments, additional constant regions are displayed.

Antibody libraries can be constructed by a number of processes. Elements of each process can be combined with those of other processes. The processes can be used such that variation is introduced into a single immunoglobulin domain (e.g., VH or VL) or into multiple immunoglobulin domains (e.g., VH and VL). The variation can be introduced into an immunoglobulin variable domain, e.g., in the region of one or more of CDR1, CDR2, CDR3, FR1, FR2, FR3, and FR4, referring to such regions of either and both of heavy and light chain variable domains. In one embodiment, variation is introduced into all three CDRs of a given variable domain. In another preferred embodiment, the variation is introduced into CDR1 and CDR2, e.g., of a heavy chain variable domain. Any combination is feasible.In one process, antibody libraries are constructed by inserting diverse oligonucleotides that encode CDRs into the corresponding regions of the nucleic acid. The oligonucleotides can be synthesized using monomeric nucleotides or trinucleotides. For example, Knappik et al. (2000) J. Mol. Biol. 296:57-86 describe a method for constructing CDR encoding oligonucleotides using trinucleotide synthesis and a template with engineered restriction sites for accepting the oligonucleotides.

In another process, an animal, e.g., a rodent, is immunized with the alpha₆p or a fragment thereof, e.g., a fragment including an epitope from the N-terminus of alpha₆p. The animal is optionally boosted with the antigen to further stimulate the response. Then spleen cells are isolated from the animal, and nucleic acid encoding VH and/or VL domains is amplified and cloned for expression in the display library.

In yet another process, antibody libraries are constructed from nucleic acid amplified from naive germline immunoglobulin genes. The amplified nucleic acid includes nucleic acid encoding the VH and/or VL domain. Sources of immunoglobulin-encoding nucleic acids are described below. Amplification can include PCR, e.g., with primers that anneal to the conserved constant region, or another amplification method.

Nucleic acid encoding immunoglobulin domains can be obtained from the immune cells of, e.g., a human, a primate, mouse, rabbit, camel, or rodent. In one example, the cells are selected for a particular property. B cells at various stages of maturity can be selected. In another example, the B cells are naïve.

In one embodiment, fluorescent-activated cell sorting (FACS) is used to sort B cells that express surface-bound IgM, IgD, or IgG molecules. Further, B cells expressing different isotypes of IgG can be isolated. In another preferred embodiment, the B or T cell is cultured in vitro. The cells can be stimulated in vitro, e.g., by culturing with feeder cells or by adding mitogens or other modulatory reagents, such as antibodies to CD40, CD40 ligand or CD20, phorbol myristate acetate, bacterial lipopolysaccharide, concanavalin A, phytohemagglutinin or pokeweed mitogen.

In still another embodiment, the cells are isolated from a subject that has an immunological disorder, e.g., systemic lupus erythematosus (SLE), rheumatoid arthritis, vasculitis, Sjogren syndrome, systemic sclerosis, or anti-phospholipid syndrome. The subject can be a human, or an animal, e.g., an animal model for the human disease, or an animal having an analogous disorder. In yet another embodiment, the cells are isolated from a transgenic non-human animal that includes a human immunoglobulin locus.

In one preferred embodiment, the cells have activated a program of somatic hypermutation. Cells can be stimulated to undergo somatic mutagenesis of immunoglobulin genes, for example, by treatment with anti-immunoglobulin, anti-CD40, and anti-CD38 antibodies (see, e.g., Bergthorsdottir et al. (2001) J Immunol. 166:2228). In another embodiment, the cells are naive.

The nucleic acid encoding an immunoglobulin variable domain can be isolated from a natural repertoire by the following exemplary method. First, RNA is isolated from the immune cell. Full-length (i.e., capped) mRNAs are separated (e.g. by degrading uncapped RNAs with calf intestinal phosphatase). The cap is then removed with tobacco acid pyrophosphatase and reverse transcription is used to produce the cDNAs.

The reverse transcription of the first (antisense) strand can be done in any manner with any suitable primer. See, e.g., de Haard et al. (1999) J. Biol. Chem 274:18218-30. The primer binding region can be constant among different immunoglobulins, e.g., in order to reverse transcribe different isotypes of immunoglobulin. The primer binding region can also be specific to a particular isotype of immunoglobulin. Typically, the primer is specific for a region that is 3′ to a sequence encoding at least one CDR. In another embodiment, poly-dT primers may be used (and may be preferred for the heavy-chain genes).

A synthetic sequence can be ligated to the 3′ end of the reverse transcribed strand. The synthetic sequence can be used as a primer binding site for binding of the forward primer during PCR amplification after reverse transcription. The use of the synthetic sequence can obviate the need to use a pool of different forward primers to fully capture the available diversity.

The variable domain-encoding gene is then amplified, e.g., using one or more rounds. If multiple rounds are used, nested primers can be used for increased fidelity. The amplified nucleic acid is then cloned into a display library vector.

Any method for amplifying nucleic acid sequences may be used for amplification. Methods that maximize, and do not bias, diversity are preferred. A variety of techniques can be used for nucleic acid amplification. The polymerase chain reaction (PCR; U.S. Pat. Nos. 4,683,195 and 4,683,202, Saiki, et al. (1985) Science 230:1350-1354) utilizes cycles of varying temperature to drive rounds of nucleic acid synthesis. Transcription-based methods utilize RNA synthesis by RNA polymerases to amplify nucleic acid (U.S. Pat. No 6,066,457; U.S. Pat. No 6,132,997; U.S. Pat. No 5,716,785; Sarkar et. al. (1989) Science 244: 331-34 ; Stofler et al. (1988) Science 239: 491). NASBA (U.S. Pat. Nos. 5,130,238; 5,409,818; and 5,554,517) utilizes cycles of transcription, reverse-transcription, and RnaseH-based degradation to amplify a DNA sample. Still other amplification methods include rolling circle amplification (RCA; U.S. Pat. Nos. 5,854,033 and 6,143,495) and strand displacement amplification (SDA; U.S. Pat. Nos. 5,455,166 and 5,624,825).

Secondary Screening Methods

After selecting candidate display library members that bind to a target, each candidate display library member can be further analyzed, e.g., to further characterize its binding properties for the target. Each candidate display library member can be subjected to one or more secondary screening assays. The assay can be for a binding property, a catalytic property, a physiological property (e.g., cytotoxicity, renal clearance, immunogenicity), a structural property (e.g., stability, conformation, oligomerization state) or another functional property. The same assay can be used repeatedly, but with varying conditions, e.g., to determine pH, ionic, or thermal sensitivities.

As appropriate, the assays can use the display library member directly, a recombinant polypeptide produced from the nucleic acid encoding a displayed polypeptide, or a synthetic peptide synthesized based on the sequence of a displayed peptide. Exemplary assays for binding properties include the following.

ELISA. Polypeptides encoded by a display library can also be screened for a binding property using an ELISA assay. For example, each polypeptide is contacted to a microtitre plate whose bottom surface has been coated with the target, e.g., a limiting amount of the target. The plate is washed with buffer to remove non-specifically bound polypeptides. Then the amount of the polypeptide bound to the plate is determined by probing the plate with an antibody that can recognize the polypeptide, e.g., a tag or constant portion of the polypeptide. The antibody is linked to an enzyme such as alkaline phosphatase, which produces a colorimetric product when appropriate substrates are provided. The polypeptide can be purified from cells or assayed in a display library format, e.g., as a fusion to a filamentous bacteriophage coat. In another version of the ELISA assay, each polypeptide of a diversity strand library is used to coat a different well of a microtitre plate. The ELISA then proceeds using a constant target molecule to query each well.

Homogeneous Binding Assays. The binding interaction of candidate polypeptide with a target can be analyzed using a homogenous assay, i.e., after all components of the assay are added, additional fluid manipulations are not required. For example, fluorescence resonance energy transfer (FRET) can be used as a homogenous assay (see, for example, Lakowicz et al., U.S. Pat. No. 5,631,169; Stavrianopoulos, et al., U.S. Pat. No. 4,868,103). A fluorophore label on the first molecule (e.g., the molecule identified in the fraction) is selected such that its emitted fluorescent energy can be absorbed by a fluorescent label on a second molecule (e.g., the target) if the second molecule is in proximity to the first molecule. The fluorescent label on the second molecule fluoresces when it absorbs to the transferred energy. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A binding event that is configured for monitoring by FRET can be conveniently measured through standard fluorometric detection means well known in the art (e.g., using a fluorimeter). By titrating the amount of the first or second binding molecule, a binding curve can be generated to estimate the equilibrium binding constant.

Another example of a homogenous assay is Alpha Screen (Packard Bioscience, Meriden Conn.). Alpha Screen uses two labeled beads. One bead generates singlet oxygen when excited by a laser. The other bead generates a light signal when singlet oxygen diffuses from the first bead and collides with it. The signal is only generated when the two beads are in proximity. One bead can be attached to the display library member, the other to the target. Signals are measured to determine the extent of binding.

The homogenous assays can be performed while the candidate polypeptide is attached to the display library vehicle, e.g., a bacteriophage.

Surface Plasmon Resonance (SPR). The binding interaction of a molecule isolated from a display library and a target can be analyzed using SPR. SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BlAcore International AB (Uppsala, Sweden).

Information from SPR can be used to provide an accurate and quantitative measure of the equilibrium dissociation constant (K_{D}), and kinetic parameters, including Kₒₙ and K_{off}, for the binding of a biomolecule to a target. Such data can be used to compare different biomolecules. For example, proteins encoded by nucleic acid selected from a library of diversity strands can be compared to identify individuals that have high affinity for the target or that have a slow K_{off}. This information can also be used to develop structure-activity relationships (SAR). For example, the kinetic and equilibrium binding parameters of matured versions of a parent protein can be compared to the parameters of the parent protein. Variant amino acids at given positions can be identified that correlate with particular binding parameters, e.g., high affinity and slow K_{off}. This information can be combined with structural modeling (e.g., using homology modeling, energy minimization, or structure determination by crystallography or NMR). As a result, an understanding of the physical interaction between the protein and its target can be formulated and used to guide other design processes.

Protein Arrays. Polypeptides identified from the display library can be immobilized on a solid support, for example, on a bead or an array. For a protein array, each of the polypeptides is immobilized at a unique address on a support. Typically, the address is a two-dimensional address. Protein arrays are described below (see, e.g., Diagnostics).

Cellular Assays. A library of candidate polypeptides (e.g., previously identified by a display library or otherwise) can be screened by transforming the library into a host cell. For example, the library can include vector nucleic acid sequences that include segments that encode the polypeptides and that direct expression, e.g., such that the polypeptides are produced within the cell, secreted from the cell, or attached to the cell surface. The cells can be screened for polypeptides that bind to the alpha₆p, e.g., as detected by a change in a cellular phenotype or a cell-mediated activity. For example, in the case of an antibody that binds to the alpha₆p, the activity may be cell or complement-mediated cytotoxicity.

In another embodiment, the library of cells is in the form of a cellular array. The cellular array can likewise be screened for any appropriate detectable activity.

Ligand Production

Standard recombinant nucleic acid methods can be used to express a protein ligand that binds to alpha₆p but that does not substantially bind to alpha₆ integrin. Generally, a nucleic acid sequence encoding the protein ligand is cloned into a nucleic acid expression vector. Of course, if the protein includes multiple polypeptide chains, each chain must be cloned into an expression vector, e.g., the same or different vectors, that are expressed in the same or different cells. If the protein is sufficiently small, i.e., the protein is a peptide of less than 50 amino acids, the protein can be synthesized using automated organic synthetic methods. Methods for producing antibodies are also provided below.

The expression vector for expressing the protein ligand can include, in addition to the segment encoding the protein ligand or fragment thereof, regulatory sequences, including for example, a promoter, operably linked to the nucleic acid(s) of interest. Large numbers of suitable vectors and promoters are known to those of skill in the art and are commercially available for generating the recombinant constructs of the present invention. The following vectors are provided by way of example. Bacterial: pBs, phagescript, PsiX174, pBluescript SK, pBs KS, pNH8a, pNH16a, pNH18a, pNH46a (Stratagene); pTrc99A, pKK223-3, pKK233-3, pDR540, and pRIT5 (Pharmacia). Eukaryotic: pWLneo, pSV2cat, pOG44, PXTI, pSG (Stratagene) pSVK3, pBPV, pMSG, and pSVL (Pharmacia). One preferred class of preferred libraries is the display library, which is described below.

Methods well known to those skilled in the art can be used to construct vectors containing a polynucleotide of the invention and appropriate transcriptional/translational control signals. These methods include in vitro recombinant DNA techniques, synthetic techniques and in vivo recombination/genetic recombination. See, for example, the techniques described in Sambrook & Russell, Molecular Cloning: A Laboratory Manual, 3^{rd} Edition, Cold Spring Harbor Laboratory, N.Y. (2001) and Ausubel et al., Current Protocols in Molecular Biology (Greene Publishing Associates and Wiley Interscience, N.Y. (1989). Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T3, T7, gpt, lambda P, and trc. Eukaryotic promoters include CMV immediate early, HSV thymidine kinase, early and late SV40, LTRs from retrovirus, mouse metallothionein-I, and various art-known tissue specific promoters.

Generally, recombinant expression vectors will include origins of replication and selectable markers permitting transformation of the host cell, e.g., the ampicillin resistance gene of E. coli and S. cerevisiae auxotrophic markers (such as URA3, LEU2, HIS3, and TRPl genes), and a promoter derived from a highly expressed gene to direct transcription of a downstream structural sequence. Such promoters can be derived from operons encoding glycolytic enzymes such as 3-phosphoglycerate kinase (PGK), a-factor, acid phosphatase, or heat shock proteins, among others. The polynucleotide of the invention is assembled in appropriate phase with translation initiation and termination sequences, and preferably, a leader sequence capable of directing secretion of translated protein into the periplasmic space or extracellular medium. Optionally, a nucleic acid of the invention can encode a fusion protein including an N-terminal identification peptide imparting desired characteristics, e.g., stabilization or simplified purification of expressed recombinant product. Useful expression-vectors for bacteria are constructed by inserting a polynucleotide of the invention together with suitable translation initiation and termination signals, optionally in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

As a representative but nonlimiting example, useful expression vectors for bacteria can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and pGEM1 (Promega, Madison, Wis., USA).

The present invention further provides host cells containing the vectors of the present invention, wherein the nucleic acid has been introduced into the host cell using known transformation, transfection or infection methods. For example, the host cells can include members of a library constructed from the diversity strand. The host cell can be a eukaryotic host cell, such as a mammalian cell, a lower eukaryotic host cell, such as a yeast cell, or the host cell can be a prokaryotic cell, such as a bacterial cell. Introduction of the recombinant construct into the host cell can be effected, for example, by calcium phosphate transfection, DEAE, dextran mediated transfection, or electroporation (Davis, L. et al. Basic Methods in Molecular Biology (1986)).

Any host/vector system can be used to identify one or more of the target elements of the present invention. These include, but are not limited to, eukaryotic hosts such as HeLa cells, CV-1 cell, COS cells, and Sf9 cells, as well as prokaryotic host such as E. coli and B. subtilis. The most preferred cells are those which do not normally express the particular reporter polypeptide or protein or which expresses the reporter polypeptide or protein at low natural level.

The host of the present invention may also be a yeast or other fungi. In yeast, a number of vectors containing constitutive or inducible promoters may be used. For reviews, see Current Protocols in Molecular Biology, Vol. 2, Ed. Ausubel et al., Greene Publish. Assoc. & Wiley Interscience, Ch. 13 (1988); Grant et al., Expression and Secretion Vectors for Yeast, in Methods in Enzymology, Ed. Wu & Grossman, Acad. Press, N.Y. 153:516-544 (1987); Glover, DNA Cloning, Vol. II, IRL Press, Wash., D.C., Ch. 3 (1986); Bitter, Heterologous Gene Expression in Yeast, in Methods in Enzymology, Eds. Berger & Kimmel, Acad. Press, N.Y. 152:673-684 (1987); and The Molecular Biology of the Yeast Saccharomyces, Eds. Strathem et al., Cold Spring Harbor Press, Vols. I and II (1982).

The host of the invention may also be a prokaryotic cell such as E. coli, other enterobacteriaceae such as Serratia marescans, bacilli, various pseudomonads, or other prokaryotes which can be transformed, transfected, infected.

The present invention further provides host cells genetically engineered to contain the polynucleotides of the invention. For example, such host cells may contain nucleic acids of the invention introduced into the host cell using known transformation, transfection or infection methods. The present invention still further provides host cells genetically engineered to express the polynucleotides of the invention, wherein such polynucleotides are in operative association with a regulatory sequence heterologous to the host cell, which drives expression of the polynucleotides in the cell.

The host cell can be a higher eukaryotic host cell, such as a mammalian cell, a lower eukaryotic host cell, such as a yeast cell, or the host cell can be a prokaryotic cell, such as a bacterial cell.

Introduction of the recombinant construct into the host cell can be effected by calcium phosphate transfection, DEAE, dextran mediated transfection, or electroporation (Davis, L. et aL, Basic Methods in Molecular Biology (1986)). The host cells containing one of polynucleotides of the invention can be used in conventional manners to produce the gene product encoded by the isolated fragment (in the case of an ORF).

Any host/vector system can be used to express one or more of the diversity strands of the present invention. These include, but are not limited to, eukaryotic hosts such as HeLa cells, CV-1 cell, COS cells, and Sf9 cells, as well as prokaryotic host such as E. coli and B. subtilis. The most preferred cells are those which do not normally express the particular polypeptide or protein or which expresses the polypeptide or protein at low natural level. Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., in Molecular Cloning: A Laboratory Manual, Second Edition, Cold Spring Harbor, N.Y. (1989), the disclosure of which is hereby incorporated by reference.

Various mammalian cell culture systems can also be employed to express recombinant protein.

Examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa and BHK cell lines. Mammalian expression vectors will comprise an origin of replication, a suitable promoter and also any necessary ribosome-binding sites, polyadenylation site, splice donor and acceptor sites, transcriptional termination sequences, and 5′ flanking nontranscribed sequences.

DNA sequences derived from the SV40 viral genome, for example, SV40 origin, early promoter, enhancer, splice, and polyadenylation sites may be used to provide the required nontranscribed genetic elements. Recombinant polypeptides and proteins produced in bacterial culture are usually isolated by initial extraction from cell pellets, followed by one or more salting-out, aqueous ion exchange or size exclusion chromatography steps. In some embodiments, the template nucleic acid also encodes a polypeptide tag, e.g., penta- or hexa-histidine. The recombinant polypeptides encoded by a library of diversity strands can then be purified using affinity chromatography.

Microbial cells employed in expression of proteins can be disrupted by any convenient method, including freeze-thaw cycling, sonication, mechanical disruption, or use of cell lysing agents. A number of types of cells may act as suitable host cells for expression of the protein. Scopes (1994) Protein Purification: Principles and Practice, New York:Springer-Verlag provides a number of general methods for purifying recombinant (and non-recombinant) proteins. The method include, e.g., ion-exchange chromatography, size-exclusion chromatography, affinity chromatography, selective precipitation, dialysis, and hydrophobic interaction chromatography. These methods can be adapted for devising a purification strategy for the anti-alpha₆p protein ligand.

Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

Alternatively, it may be possible to produce the protein in lower eukaryotes such as yeast or in prokaryotes such as bacteria. Potentially suitable yeast strains include Saccharomyces cerevisiae, Schizosaccharomyces pombe, Kluyveromyces strains, Candida, Pichia or any yeast strain capable of expressing heterologous proteins. Potentially suitable bacterial strains include Escherichia coli, Bacillus subtilis, Salmonella typhimurium, or any bacterial strain capable of expressing heterologous proteins. If the protein is made in yeast or bacteria, it may be necessary to modify the protein produced therein, for example by phosphorylation or glycosylation of the appropriate sites, in order to obtain the functional protein. Such covalent attachments may be accomplished using known chemical or enzymatic methods. In another embodiment of the present invention, cells and tissues may be engineered to express an endogenous gene comprising the polynucleotides of the invention under the control of inducible regulatory elements, in which case the regulatory sequences of the endogenous gene may be replaced by homologous recombination. As described herein, gene targeting can be used to replace a gene's existing regulatory region with a regulatory sequence isolated from a different gene or a novel regulatory sequence synthesized by genetic engineering methods.

Such regulatory sequences may be comprised of promoters, enhancers, scaffold-attachment regions, negative regulatory elements, transcriptional initiation sites, regulatory protein binding sites or combinations of said sequences. Alternatively, sequences which affect the structure or stability of the RNA or protein produced may be replaced, removed, added, or otherwise modified by targeting, including polyadenylation signals. mRNA stability elements, splice sites, leader sequences for enhancing or modifying transport or secretion properties of the protein, or other sequences which alter or improve the function or stability of protein or RNA molecules.

Antibody Production. Some antibodies, e.g., Fabs, can be produced in bacterial cells, e.g., E. coli cells. For example, if the Fab is encoded by sequences in a phage display vector that includes a suppressible stop codon between the display entity and a bacteriophage protein (or fragment thereof), the vector nucleic acid can be shuffled into a bacterial cell that cannot suppress a stop codon. In this case, the Fab is not fused to the gene III protein and is secreted into the media.

Antibodies can also be produced in eukaryotic cells. In one embodiment, the antibodies (e.g., scFv's) are expressed in a yeast cell such as Pichia (see, e.g., Powers et al. (2001) J Immunol Methods. 251:123-35), Hanseula, or Saccharomyces.

In one preferred embodiment, antibodies are produced in mammalian cells. Preferred mammalian host cells for expressing the clone antibodies or antigen-binding fragments thereof include Chinese Hamster Ovary (CHO cells) (including dhfr− CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NS0 myeloma cells and SP2 cells, COS cells, and a cell from a transgenic animal, e.g., a transgenic mammal. For example, the cell is a mammary epithelial cell.

In addition to the nucleic acid sequence encoding the diversified immunoglobulin domain, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

In an exemplary system for recombinant expression of an antibody, or antigen-binding portion thereof, of the invention, a recombinant expression vector encoding both the antibody heavy chain and the antibody light chain is introduced into dhfr− CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. For example, some antibodies can be isolated by affinity chromatography with a Protein A or Protein G.

For antibodies that include an Fc domain, the antibody production system preferably synthesizes antibodies in which the Fc region is glycosylated. For example, the Fe domain of IgG molecules is glycosylated at asparagine 297 in the CH2 domain. This asparagine is the site for modification with biantennary-type oligosaccharides. It has been demonstrated that this glycosylation is required for effector functions mediated by Fcγ receptors and complement C1q (Burton and Woof (1992) Adv. Immunol. 51:1-84; Jefferis et al. (1998) Immunol. Rev. 163:59-76). In a preferred embodiment, the Fc domain is produced in a mammalian expression system that appropriately glycosylates the residue corresponding to asparagine 297. The Fc domain can also include other eukaryotic post-translational modifications.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly.

Producing a Truncated Alpha 6 Integrin.

The invention also provides for a method of producing an N-terminal truncated alpha6 variant peptide. The method includes culturing a host cell that contains a nucleic acid encoding the polypeptide under the appropriate conditions. For example, a mouse cell, such as from a 291, O3C or O3R cell line, can be maintained in calcium conditions ranging from 0.01 to 2.0 mM, preferably 0.04-1.4 mM. For a mouse 291 cell in particular, terminal differentiation induced by high calcium levels (0.8-2.0 mM, preferably 1.0-1.5 mM, more preferably 1.4 mM) can increase production of an alpha6 integrin variant polypeptide, particularly an alpha6p polypeptide.

A human cell can also be cultured to produce an alpha6 integrin variant polypeptide. A human cell line can be cultured, for example, at 37° C. in a humidified atmosphere of 95% air and 5% CO₂. Human cells can be cultured in medium, such as Iscove's modified Dulbecco's medium (Invitrogen Corporation, Carlsbad, Calif.; formerly Life Technologies, Inc.) plus 10% fetal bovine serum; Dulbecco's modified Eagle's medium (Invitrogen Corporation, Carlsbad, Calif.; formerly Life Technologies, Inc.) plus 10% fetal bovine serum; Dulbecco's modified Eagle's medium plus 5% nonessential amino acids, 5% L-glutamine, 5% sodium pyruvate, 10% fetal bovine serum; or PrEGM bullet kit medium (Clonetics, San Diego, Calif.). Optimum culture conditions can depend on the type, e.g., origin, of the cell line and can be determined empirically.

Standard recombinant nucleic acid methods can be used to express a protein that includes a truncated alpha6 integrin, e.g., as described for protein ligands above. Generally, a nucleic acid sequence encoding the protein is cloned into a nucleic acid expression vector. In a typical embodiment, the nucleic acid does not include sequences encoding amino acids sequence segments of the full length alpha6 integrin that are absent from the truncated alpha6 integrin. Information about the genetic code can be used to design an appropriate coding nucleic acid, e.g., by fusion to a signal sequence so that cleavage of the signal sequence will produce the truncated alpha6 integrin (e.g., at least the extracellular domain) in a secreted or transmembrane form. The expression vector for expressing the protein can include, in addition to the segment encoding the protein or fragment thereof, regulatory sequences, including for example, a promoter, operably linked to the nucleic acid(s) of interest.

In still another method, a cell that produces integrin alpha6, e.g., from an endogenous gene or a heterologous gene (e.g., a gene that includes an alpha6 coding sequence and an operably linked promoter, e.g., an inducible promoter) is cultured so that the integrin is produced. The cell is contacted with a protease, e.g., urokinase, under conditions in which the protease cleaves the integrin. The cell can then be used, e.g., for ligand screening, wound healing, or for an extraction, e.g., to obtain a cell-free preparation of a truncated alpha6 integrin.

Pharmaceutical Compositions

In another aspect, the present invention provides compositions, e.g., pharmaceutically acceptable compositions, which include an anti-alpha₆p ligand, e.g., an antibody molecule, or other polypeptide or peptide identified as binding to alpha₆p described herein, formulated together with a pharmaceutically acceptable carrier. As used herein, “pharmaceutical compositions” encompass labeled ligands for in vivo imaging as well as therapeutic compositions.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., protein ligand may be coated in a material to protect the compound from the action of acids and other natural conditions that may inactivate the compound.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for administration of humans with antibodies. The preferred mode of administration is parenteral (e.g., intravenous, subcutaneous, intraperitoneal, intramuscular). In a preferred embodiment, the anti-alpha₆p ligand is administered by intravenous infusion or injection. In another preferred embodiment, the anti-alpha₆p ligand is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Pharmaceutical compositions typically must be sterile and stable under the conditions of manufacture and storage. A pharmaceutical composition can also be tested to insure it meets regulatory and industry standards for administration. For example, endotoxin levels in the preparation can be tested using the Limulus amebocyte lysate assay (e.g., using the kit from Bio Whittaker lot #7L3790, sensitivity 0.125 EU/mL) according to the USP 24/NF 19 methods. Sterility of pharmaceutical compositions can be determined using thioglycollate medium according to the USP 24/NF 19 methods. For example, the preparation is used to inoculate the thioglycollate medium and incubated at 35° C. for 14 or more days. The medium is inspected periodically to detect growth of a microorganism.

The composition can be formulated as a solution, microemulsion, dispersion, liposome, or other ordered structure suitable to high drug concentration. Sterile injectable solutions can be prepared by incorporating the active compound (i.e., the ligand) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The anti-alpha₆p protein ligands of the present invention can be administered by a variety of methods known in the art, although for many applications, the preferred route/mode of administration is intravenous injection or infusion. For example, for therapeutic applications, the anti-alpha₆p ligand can be administered by intravenous infusion at a rate of less than 30, 20, 10, 5, or 1 mg/min to reach a dose of about 1 to 100 mg/m² or 7 to 25 mg/m². The route and/or mode of administration will vary depending upon the desired results. In certain embodiments, the active compound may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

In certain embodiments, the ligand may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Pharmaceutical compositions can be administered with medical devices known in the art. For example, in a preferred embodiment, a pharmaceutical composition of the invention can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Of course, many other such implants, delivery systems, and modules are also known.

In certain embodiments, the compounds of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties, which are selectively transported into specific cells or organs, thus enhance targeted drug delivery (see, e.g., V. V. Ranade (1989) J. Clin. Pharmacol. 29:685).

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of an antibody of the invention is 0.1-20 mg/kg, more preferably 1-10 mg/kg. The anti-alpha₆p antibody can be administered by intravenous infusion at a rate of less than 30, 20, 10, 5, or 1 mg/min to reach a dose of about 1 to 100 mg/m² or about 5 to 30 mg/m². For ligands smaller in molecular weight than an antibody, appropriate amounts can be proportionally less. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the invention may include a “therapeutically effective amount” or a “prophylactically effective amount” of an anti-alpha₆p ligand of the invention. A “therapeutically effective amount” refers to an amount effective, at dosages and for periods of time necessary, to achieve the desired therapeutic result. A therapeutically effective amount of the composition may vary according to factors such as the disease state, age, sex, and weight of the individual, and the ability of the protein ligand to elicit a desired response in the individual. A therapeutically effective amount is also one in which any toxic or detrimental effects of the composition is outweighed by the therapeutically beneficial effects. A “therapeutically effective dosage” preferably inhibits a measurable parameter, e.g., tumor growth rate by at least about 20%, more preferably by at least about 40%, even more preferably by at least about 60%, and still more preferably by at least about 80% relative to untreated subjects. The ability of a compound to inhibit a measurable parameter, e.g., cancer, can be evaluated in an animal model system predictive of efficacy in human tumors. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the invention are kits comprising the protein ligand that binds to alpha₆p and instructions for use, e.g., treatment, prophylactic, or diagnostic use. In one embodiment, the instructions for diagnostic applications include the use of the anti-alpha₆p ligand (e.g., antibody or antigen-binding fragment thereof, or other polypeptide or peptide) to detect alpha₆p, in vitro, e.g., in a sample, e.g., a biopsy or cells from a patient having a cancer or neoplastic disorder, or in vivo. In another embodiment, the instructions for therapeutic applications include suggested dosages and/or modes of administration in a patient with a cancer or neoplastic disorder. The kit can further contain a least one additional reagent, such as a diagnostic or therapeutic agent, e.g., a diagnostic or therapeutic agent as described herein, and/or one or more additional anti-alpha₆p ligands, formulated as appropriate, in one or more separate pharmaceutical preparations.

Treatments

Protein ligands that bind to alpha₆p and that do not substantially bind to full-length alpha₆ integrin, e.g., ligands described herein, have therapeutic and prophylactic utilities. For example, these ligands can be administered to cells in culture, e.g. in vitro or ex vivo, or in a subject, e.g., in vivo, to treat, prevent, and/or diagnose a variety of disorders, such as cancer, e.g., prostate cancer.

As used herein, the term “treat” or “treatment” is defined as the application or administration of an anti-alpha₆p ligand, e.g., antibody, alone or in combination with, a second agent to a subject, e.g., a patient, or application or administration of the agent to an isolated tissue or cell, e.g., cell line, from a subject, e.g., a patient, who has a disorder (e.g., a disorder as described herein), a symptom of a disorder or a predisposition toward a disorder, with the purpose to cure, heal, alleviate, relieve, alter, remedy, ameliorate, improve or affect the disorder, the symptoms of the disorder or the predisposition toward the disorder. Treating a cell refers to the inhibition, ablation, killing of a cell in vitro or in vivo, or otherwise reducing capacity of a cell, e.g., an aberrant cell, to mediate a disorder, e.g., a disorder as described herein (e.g., a cancerous disorder). In one embodiment, “treating a cell” refers to a reduction in the activity and/or proliferation of a cell, e.g., a hyperproliferative cell. Such reduction does not necessarily indicate a total elimination of the cell, but a reduction, e.g., a statistically significant reduction, in the activity or the number of the cell.

As used herein, an amount of an anti-alpha₆p specific ligand effective to treat a disorder, or a “therapeutically effective amount” refers to an amount of the ligand which is effective, upon single or multiple dose administration to a subject, in treating a cell, e.g., a cancer cell (e.g., a alpha₆p-expressing cancer cell, e.g., prostate cancer cell), or in prolonging curing, alleviating, relieving or improving a subject with a disorder as described herein beyond that expected in the absence of such treatment. As used herein, “inhibiting the growth” of the neoplasm refers to slowing, interrupting, arresting or stopping its growth and metastases and does not necessarily indicate a total elimination of the neoplastic growth.

As used herein, an amount of an anti-alpha₆p ligand effective to prevent a disorder, or a “a prophylactically effective amount” of the ligand refers to an amount of an anti-alpha₆p ligand, e.g., an anti-alpha₆p antibody described herein, which is effective, upon single- or multiple-dose administration to the subject, in preventing or delaying the occurrence of the onset or recurrence of a disorder, e.g., a cancer.

The terms “induce”, “inhibit”, “potentiate”, “elevate”, “increase”, “decrease” or the like, e.g., which denote quantitative differences between two states, refer to a difference, e.g., a statistically significant difference, between the two states. For example, “an amount effective to inhibit the proliferation of the alpha₆p-expressing hyperproliferative cells” means that the rate of growth of the cells will be different, e.g., statistically significantly different, from the untreated cells.

As used herein, the term “subject” is intended to include human and non-human animals. Preferred human animals include a human patient having a disorder characterized by abnormal cell proliferation or cell differentiation. The term “non-human animals” of the invention includes all vertebrates, e.g., non-mammals (such as chickens, amphibians, reptiles) and mammals, such as non-human primates, sheep, dog, cow, pig, etc.

In one embodiment, the subject is a human subject. Alternatively, the subject can be a mammal expressing a alpha₆p-like antigen with which an antibody of the invention cross-reacts. A protein ligand of the invention can be administered to a human subject for therapeutic purposes (discussed further below). Moreover, an anti-alpha₆p specific ligand can be administered to a non-human mammal expressing the alpha₆p-like antigen to which the ligand binds (e.g., a primate, pig or mouse) for veterinary purposes or as an animal model of human disease. Regarding the latter, such animal models may be useful for evaluating the therapeutic efficacy of the ligand (e.g., testing of dosages and time courses of administration).

In one embodiment, the invention provides a method of treating (e.g., ablating or killing) a cell (e.g., a non-cancerous cell, e.g., a normal, benign or hyperplastic cell, or a cancerous cell, e.g., a malignant cell, e.g., cell found in a solid tumor, a soft tissue tumor, or a metastatic lesion (e.g., a cell found in prostate, renal, urothelial, colonic, rectal, pulmonary, breast or hepatic, cancers and/or metastasis). Methods of the invention include the steps of contacting the cell with an anti-alpha₆p ligand, e.g., an anti-alpha₆p antibody described herein, in an amount sufficient to treat, e.g., ablate or kill, the cell.

The subject method can be used on cells in culture, e.g. in vitro or ex vivo. For example, cancerous or metastatic cells (e.g., prostate, renal, urothelial, colon, rectal, lung, breast, ovarian, prostatic, or liver cancerous or metastatic cells) can be cultured in vitro in culture medium and the contacting step can be effected by adding the anti-alpha₆p ligand to the culture medium. The method can be performed on cells (e.g., cancerous or metastatic cells) present in a subject, as part of an in vivo (e.g., therapeutic or prophylactic) protocol. For in vivo embodiments, the contacting step is effected in a subject and includes administering the anti-alpha₆p ligand to the subject under conditions effective to permit both binding of the ligand to the cell and the treating, e.g., the killing or ablating of the cell.

The method can be used to treat a cancer. As used herein, the terms “cancer”, “hyperproliferative”, “malignant”, and “neoplastic” are used interchangeably, and refer to those cells an abnormal state or condition characterized by rapid proliferation or neoplasm. The terms include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth.

The common medical meaning of the term “neoplasia” refers to “new cell growth” that results as a loss of responsiveness to normal growth controls, e.g. to neoplastic cell growth. A “hyperplasia” refers to cells undergoing an abnormally high rate of growth. However, as used herein, the terms neoplasia and hyperplasia can be used interchangeably, as their context will reveal, referring generally to cells experiencing abnormal cell growth rates. Neoplasias and hyperplasias include “tumors,” which may be benign, premalignant or malignant.

Examples of cancerous disorders include, but are not limited to, solid tumors, soft tissue tumors, and metastatic lesions. Examples of solid tumors include malignancies, e.g., sarcomas, adenocarcinomas, and carcinomas, of the various organ systems, such as those affecting lung, breast, lymphoid, gastrointestinal (e.g., colon), and genitourinary tract (e.g., renal, urothelial cells), pharynx, prostate, ovary as well as adenocarcinomas which include malignancies such as most colon cancers, rectal cancer, renal-cell carcinoma, liver cancer, non-small cell carcinoma of the lung, cancer of the small intestine and so forth. Metastatic lesions of the aforementioned cancers can also be treated or prevented using the methods and compositions of the invention.

The subject method can be useful in treating malignancies of the various organ systems, such as those affecting lung, breast, lymphoid, gastrointestinal (e.g., colon), and genitourinary tract, prostate, ovary, pharynx, as well as adenocarcinomas which include malignancies such as most colon cancers, renal-cell carcinoma, prostate cancer and/or testicular tumors, non-small cell carcinoma of the lung, cancer of the small intestine and cancer of the esophagus. Exemplary solid tumors that can be treated include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

The term “carcinoma” is recognized by those skilled in the art and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is recognized by those skilled in the art and refers to malignant tumors of mesenchymal derivation.

The subject method can also be used to inhibit the proliferation of hyperplastic/neoplastic cells of hematopoietic origin, e.g., arising from myeloid, lymphoid or erythroid lineages, or precursor cells thereof. For instance, the present invention contemplates the treatment of various myeloid disorders including, but not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97). Lymphoid malignancies which maybe treated by the subject method include, but are not limited to acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas contemplated by the treatment method of the present invention include, but are not limited to, non-Hodgkin's lymphoma and variants thereof, peripheral T-cell lymphomas, adult T-cell leukemia/lymphoma (ATL), cutaneous T-cell lymphoma (CTCL), large granular lymphocytic leukemia (LGF) and Hodgkin's disease.

Methods of administering anti-alpha₆p ligands are described in “Pharmaceutical Compositions”. Suitable dosages of the molecules used will depend on the age and weight of the subject and the particular drug used. The ligands can be used as competitive agents to inhibit, reduce an undesirable interaction, e.g., between a natural or pathological agent and the alpha₆p.

In one embodiment, the anti-alpha₆p ligands are used to kill or ablate cancerous cells and normal, benign hyperplastic, and cancerous cells in vivo. The ligands can be used by themselves or conjugated to an agent, e.g., a cytotoxic drug, radioisotope. This method includes: administering the ligand alone or attached to a cytotoxic drug, to a subject requiring such treatment.

The terms “cytotoxic agent” and “cytostatic agent” and “anti-tumor agent” are used interchangeably herein and refer to agents that have the property of inhibiting the growth or proliferation (e.g., a cytostatic agent), or inducing the killing, of hyperproliferative cells, e.g., an aberrant cancer cell. In cancer therapeutic embodiment, the term “cytotoxic agent” is used interchangeably with the terms “anti-cancer” or “anti-tumor” to mean an agent, which inhibits the development or progression of a neoplasm, particularly a solid tumor, a soft tissue tumor, or a metastatic lesion.

Nonlimiting examples of anti-cancer agents include, e.g., antimicrotubule agents, topoisomerase inhibitors, antimetabolites, mitotic inhibitors, alkylating agents, intercalating agents, agents capable of interfering with a signal transduction pathway, agents that promote apoptosis, radiation, and antibodies against other tumor-associated antigens (including naked antibodies, immunotoxins and radioconjugates). Examples of the particular classes of anti-cancer agents are provided in detail as follows: antitubulin/antimicrotubule, e.g., paclitaxel, taxol, vincristine, vinblastine, vindesine, vinorelbin, taxotere; topoisomerase I inhibitors, e.g., topotecan, camptothecin, doxorubicin, etoposide, mitoxantrone, daunorubicin, idarubicin, teniposide, amsacrine, epirubicin, merbarone, piroxantrone hydrochloride; antimetabolites, e.g., 5-fluorouracil (5-FU), methotrexate, 6-mercaptopurine, 6-thioguanine, fludarabine phosphate, cytarabine/Ara-C, trimetrexate, gemcitabine, acivicin, alanosine, pyrazofurin, N-Phosphoracetyl-L-Asparate=PALA, pentostatin, 5-azacitidine, 5-Aza 2′-deoxycytidine, ara-A, cladribine, 5-fluorouridine, FUDR, tiazofurin, N-[5-[N-(3,4-dihydro-2-methyl-4-oxoquinazolin-6-ylmethyl)-N-methylamino]-2-thenoyl]-L-glutamic acid; alkylating agents, e.g., cisplatin, carboplatin, mitomycin C, BCNU=Carmustine, melphalan, thiotepa, busulfan, chlorambucil, plicamycin, dacarbazine, ifosfamide phosphate, cyclophosphamide, nitrogen mustard, uracil mustard, pipobroman, 4-ipomeanol; agents acting via other mechanisms of action, e.g., dihydrolenperone, spiromustine, and desipeptide; biological response modifiers, e.g., to enhance anti-tumor responses, such as interferon; apoptotic agents, such as actinomycin D; and anti-hormones, for example anti-estrogens such as tamoxifen or, for example antiandrogens such as 4′-cyano-3-(4-fluorophenylsulphonyl)-2-hydroxy-2-methyl-3′-(trifluoromethyl) propionanilide.

Since the anti-alpha₆p ligands may recognize normal cells in addition to hyperproliferating cells, any such cells to which the ligands bind are destroyed. Alternatively, the ligands bind to cells in the vicinity of the cancerous cells and kill them, thus indirectly attacking the cancerous cells, which may rely on surrounding cells for nutrients, growth signals and so forth. Thus, the anti-alpha₆p ligands (e.g., modified with a cytotoxin) can selectively kill or ablate cells in cancerous tissue (including the cancerous cells themselves).

The ligands may be used to deliver a variety of cytotoxic drugs including therapeutic drugs, a compound emitting radiation, molecules of plants, fungal, or bacterial origin, biological proteins, and mixtures thereof. The cytotoxic drugs can be intracellularly acting cytotoxic drugs, such as short-range radiation emitters, including, for example, short-range, high-energy α-emitters, as described herein.

Enzymatically active toxins and fragments thereof are exemplified by diphtheria toxin A fragment, nonbinding active fragments of diphtheria toxin, exotoxin A (from Pseudomonas aeruginosa), ricin A chain, abrin A chain, modeccin A chain, α-sacrin, certain Aleurites fordii proteins, certain Dianthin proteins, Phytolacca americana proteins (PAP, PAPII and PAP-S), Morodica charantia inhibitor, curcin, crotin, Saponaria officinalis inhibitor, gelonin, mitogillin, restrictocin, phenomycin, and enomycin. Procedures for preparing enzymatically active polypeptides of the immunotoxins are described in W084/03508 and W085/03508, which are hereby incorporated by reference, and in the appended Examples below. Examples of cytotoxic moieties that can be conjugated to the antibodies include adriamycin, chlorambucil, daunomycin, methotrexate, neocarzinostatin, and platinum.

In the case of polypeptide toxins, recombinant nucleic acid techniques can be used to construct a nucleic acid that encodes the ligand (or a polypeptide component thereof) and the cytotoxin (or a polypeptide component thereof) as translational fusions. The recombinant nucleic acid is then expressed, e.g., in cells and the encoded fusion polypeptide isolated.

Procedures for conjugating protein ligands (e.g., antibodies) with the cytotoxic agents have been previously described. Procedures for conjugating chlorambucil with antibodies are described by Flechner (1973) European Journal of Cancer, 9:741-745; Ghose et al. (1972) British Medical Journal 3:495-499; and Szekerke, et al. (1972) Neoplasma 19:211-215, which are hereby incorporated by reference. Procedures for conjugating daunomycin and adriamycin to antibodies are described by Hurwitz, E. et al. (1975) Cancer Research 35:1175-1181 and Arnon et al. (1982) Cancer Surveys 1:429-449, which are hereby incorporated by reference. Procedures for preparing antibody-ricin conjugates are described in U.S. Pat. No. 4,414,148 and by Osawa et al. (1982) Cancer Surveys 1:373-388 and the references cited therein, which are hereby incorporated by reference. Coupling procedures as also described in EP 86309516.2, which is hereby incorporated by reference.

To kill or ablate normal, benign hyperplastic, or cancerous cells, a first protein ligand is conjugated with a prodrug, which is activated only when in close proximity with a prodrug activator. The prodrug activator is conjugated with a second protein ligand, preferably one that binds to a non-competing site on the target molecule. Whether two protein ligands bind to competing or non-competing binding sites can be determined by conventional competitive binding assays. Drug-prodrug pairs suitable for use in the practice of the present invention are described in Blakely et al. (1996) Cancer Research 56:3287-3292.

Alternatively, the anti-alpha₆p ligand can be coupled to high energy radiation emitters, for example, a radioisotope, such as ¹³¹I, a γ-emitter, which, when localized at the tumor site, results in a killing of several cell diameters. See, e.g., S. E. Order, “Analysis, Results, and Future Prospective of the Therapeutic Use of Radiolabeled Antibody in Cancer Therapy,” Monoclonal Antibodiesfor Cancer Detection and Therapy, R. W. Baldwin et al. (eds.), pp 303-316 (Academic Press 1985). Other suitable radioisotopes include α-emitters, such as ²¹²Bi, ²¹³Bi, and ²¹¹At, and β-emitters, such as ¹⁸⁶Re and ⁹⁰Y. Moreover, Lu¹¹⁷ may also be used as both an imaging and cytotoxic agent.

Radioimmunotherapy (RIT) using antibodies labeled with ¹³¹I, ⁹⁰Y, and ¹⁷⁷Lu is under intense clinical investigation. There are significant differences in the physical characteristics of these three nuclides and as a result, the choice of radionuclide is very critical in order to deliver maximum radiation dose to the tumor. The higher beta energy particles of ⁹⁰Y may be good for bulky tumors. The relatively low energy beta particles of ¹³¹I are ideal, but in vivo dehalogenation of radioiodinated molecules is a major disadvantage for internalizing antibody. In contrast, ¹⁷⁷Lu has low energy beta particle with only 0.2-0.3 mm range and delivers much lower radiation dose to bone marrow compared to ⁹⁰Y. In addition, due to longer physical half-life (compared to ⁹⁰Y), the tumor residence times are higher. As a result, higher activities (more mCi amounts) of ¹⁷⁷Lu labeled agents can be administered with comparatively less radiation dose to marrow. There have been several clinical studies investigating the use of ¹⁷⁷Lu labeled antibodies in the treatment of various cancers. (Mulligan et al. (1995) Clin Cancer Res. 1: 1447-1454; Meredith et al. (1996) J Nucl Med 37:1491-1496; Alvarez et al. (1997) Gynecologic Oncology 65:94-101).

The anti-alpha₆p ligands can be used directly in vivo to eliminate antigen-expressing cells via natural complement-dependent cytotoxicity (CDC) or antibody-dependent cellular cytotoxicity (ADCC). The protein ligands of the invention, can include complement binding effector domain, such as the Fc portions from IgG1, −2, or −3 or corresponding portions of IgM which bind complement. In one embodiment, a population of target cells is ex vivo treated with a binding agent of the invention and appropriate effector cells. The treatment can be supplemented by the addition of complement or serum containing complement. Further, phagocytosis of target cells coated with a protein ligand of the invention can be improved by binding of complement proteins. In another embodiment target, cells coated with the protein ligand which includes a complement binding effector domain are lysed by complement.

Also encompassed by the present invention is a method of killing or ablating which involves using the anti-alpha₆p ligand for prophylaxis. For example, these materials can be used to prevent or delay development or progression of cancers.

Use of the therapeutic methods of the present invention to treat cancers has a number of benefits. Since the protein ligands specifically recognize alpha₆p (and do not substantially recognize alpha₆ integrin), other tissue is spared and high levels of the agent are delivered directly to the site where therapy is required. Treatment in accordance with the present invention can be effectively monitored with clinical parameters. Alternatively, these parameters can be used to indicate when such treatment should be employed.

Anti-alpha₆p ligands of the invention can be administered in combination with one or more of the existing modalities for treating cancers, including, but not limited to: surgery; radiation therapy, and chemotherapy.

Diagnostic Uses

Protein ligands that bind to alpha₆p, e.g., ligands identified by the methods described herein, have in vitro and in vivo diagnostic, therapeutic and prophylactic utilities.

In one aspect, the present invention provides a diagnostic method for detecting the presence of alpha₆p, in vitro (e.g., a biological sample, such as tissue, biopsy, e.g., a cancerous tissue) or in vivo (e.g., in vivo imaging in a subject).

The method includes: (i) contacting a sample with anti-alpha₆p ligand; and (ii) detecting formation of a complex between the anti-alpha₆p ligand and the sample. The method can also include contacting a reference sample (e.g., a control sample) with the ligand, and determining the extent of formation of the complex between the ligand and the sample relative to the same for the reference sample. A change, e.g., a statistically significant change, in the formation of the complex in the sample or subject relative to the control sample or subject can be indicative of the presence of alpha₆p in the sample.

Another method includes: (i) administering the anti-alpha₆p ligand to a subject; and (iii) detecting formation of a complex between the anti-alpha₆p ligand, and the subject. The detecting can include determining location or time of formation of the complex.

The anti-alpha₆p ligand can be directly or indirectly labeled with a detectable substance to facilitate detection of the bound or unbound antibody. Suitable detectable substances include various enzymes, prosthetic groups, fluorescent materials, luminescent materials and radioactive materials.

Complex formation between the anti-alpha₆p specific ligand and alpha₆p can be detected by measuring or visualizing either the ligand bound to the alpha₆p or unbound ligand. Conventional detection assays can be used, e.g., an enzyme-linked immunosorbent assays (ELISA), a radioimmunoassay (RIA) or tissue immunohistochemistry. Further to labeling the anti-alpha₆p ligand, the presence of alpha₆p can be assayed in a sample by a competition immunoassay utilizing standards labeled with a detectable substance and an unlabeled anti-alpha₆p ligand. In one example of this assay, the biological sample, the labeled standards and the alpha₆p binding agent are combined and the amount of labeled standard bound to the unlabeled ligand is determined. The amount of alpha₆p in the sample is inversely proportional to the amount of labeled standard bound to the alpha₆p binding agent.

Fluorophore and chromophore labeled protein ligands can be prepared. Since antibodies and other proteins absorb light having wavelengths up to about 310 nm, the fluorescent moieties should be selected to have substantial absorption at wavelengths above 310 nm and preferably above 400 nm. A variety of suitable fluorescers and chromophores are described by Stryer (1968) Science 162:526 and Brand et al. (1972) Annual Review of Biochemistry 41:843-868. The protein ligands can be labeled with fluorescent chromophore groups by conventional procedures such as those disclosed in U.S. Pat. Nos. 3,940,475, 4,289,747, and 4,376,110. One group of fluorescers having a number of the desirable properties described above is the xanthene dyes, which include the fluoresceins and rhodamines. Another group of fluorescent compounds are the naphthylamines. Once labeled with a fluorophore or chromophore, the protein ligand can be used to detect the presence or localization of the alpha₆p in a sample, e.g., using fluorescent microscopy (such as confocal or deconvolution microscopy).

Histological Analysis. Immunohistochemistry can be performed using the protein ligands described herein. For example, in the case of an antibody, the antibody can synthesized with a label (such as a purification or epitope tag), or can be detectably labeled, e.g., by conjugating a label or label-binding group. For example, a chelator can be attached to the antibody. The antibody is then contacted to a histological preparation, e.g., a fixed section of tissue that is on a microscope slide. After incubation for binding, the preparation is washed to remove unbound antibody. The preparation is then analyzed, e.g., using microscopy, to identify if the antibody bound to the preparation.

Of course, the antibody (or other polypeptide or peptide) can be unlabeled at the time of binding. After binding and washing, the antibody is labeled in order to render it detectable.

Protein Arrays. The anti-alpha₆p ligand can also be immobilized on a protein array. The protein array can be used as a diagnostic tool, e.g., to screen medical samples (such as isolated cells, blood, sera, biopsies, and the like). Of course, the protein array can also include other ligands, e.g., that bind to the alpha₆p or to other target molecules, such as other integrins.

Methods of producing polypeptide arrays are described, e.g., in De Wildt et al. (2000) Nat. Biotechnol 18:989-994; Lueking et al. (1999) Anal. Biochem. 270:103-111; Ge (2000) Nucleic Acids Res. 28:e3, I-VII; MacBeath and Schreiber (2000) Science 289:1760-1763; WO 01/40803 and WO 99/51773A1. Polypeptides for the array can be spotted at high speed, e.g., using commercially available robotic apparati, e.g., from Genetic MicroSystems or BioRobotics. The array substrate can be, for example, nitrocellulose, plastic, glass, e.g., surface-modified glass. The array can also include a porous matrix, e.g., acrylamide, agarose, or another polymer.

For example, the array can be an array of antibodies, e.g., as described in De Wildt, supra. Cells that produce the protein ligands can be grown on a filter in an arrayed format. Polypeptide production is induced, and the expressed polypeptides are immobilized to the filter at the location of the cell.

A protein array can be contacted with a labeled target to determine the extent of binding of the target to each immobilized polypeptide from the diversity strand library. If the target is unlabeled, a sandwich method can be used, e.g., using a labeled probed, to detect binding of the unlabeled target.

Information about the extent of binding at each address of the array can be stored as a profile, e.g., in a computer database. The protein array can be produced in replicates and used to compare binding profiles, e.g., of a target and a non-target. Thus, protein arrays can be used to identify individual members of the diversity strand library that have desired binding properties with respect to one or more molecules.

FACS. (Fluorescent Activated Cell Sorting). The anti-alpha₆p specific ligand can be used to label cells, e.g., cells in a sample (e.g., a patient sample). The ligand is also attached (or attachable) to a fluorescent compound. The cells can then be sorted using fluorescent activated cell sorted (e.g., using a sorter available from Becton Dickinson Immunocytometry Systems, San Jose Calif.; see also U.S. Pat. No. 5,627,037; 5,030,002; and 5,137,809). As cells pass through the sorter, a laser beam excites the fluorescent compound while a detector counts cells that pass through and determines whether a fluorescent compound is attached to the cell by detecting fluorescence. The amount of label bound to each cell can be quantified and analyzed to characterize the sample.

The sorter can also deflect the cell and separate cells bound by the ligand from those cells not bound by the ligand. The separated cells can be cultured and/or characterized.

In vivo Imaging. In still another embodiment, the invention provides a method for detecting the presence of a alpha6p-expressing cancerous tissues in vivo. The method includes (i) administering to a subject (e.g., a patient having a cancer or neoplastic disorder) an anti-alpha₆p antibody, conjugated to a detectable marker; (ii) exposing the subject to a means for detecting said detectable marker to the alpha₆p-expressing tissues or cells. For example, the subject is imaged, e.g., by NMR or other tomographic means.

Examples of labels useful for diagnostic imaging in accordance with the present invention include radiolabels such as ¹³¹I, ¹¹¹In, ¹²³I, ^{99m}Tc, ³²P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed. The protein ligand can be labeled with such reagents using known techniques. For example, see Wensel and Meares (1983) Radioimmunoimaging and Radioimmunotherapy, Elsevier, New York for techniques relating to the radiolabeling of antibodies and D. Colcher et al. (1986) Meth. Enzymol. 121: 802-816.

A radiolabeled ligand of this invention can also be used for in vitro diagnostic tests. The specific activity of a isotopically-labeled ligand depends upon the half-life, the isotopic purity of the radioactive label, and how the label is incorporated into the antibody.

Procedures for labeling polypeptides with the radioactive isotopes (such as ¹⁴C, ³H, ³⁵S, ¹²⁵I, ³²P, ¹³¹I) are generally known. For example, tritium labeling procedures are described in U.S. Pat. No. 4,302,438. Iodinating, tritium labeling, and ³⁵S labeling procedures, e.g., as adapted for murine monoclonal antibodies, are described, e.g., by Goding, J. W. (Monoclonal antibodies: principles and practice: production and application of monoclonal antibodies in cell biology, biochemistry, and immunology 2nd ed. London; Orlando: Academic Press, 1986. pp 124-126) and the references cited therein. Other procedures for iodinating polypeptides, such as antibodies, are described by Hunter and Greenwood (1962) Nature 144:945, David et al. (1974) Biochemistry 13:1014-1021, and U.S. Pat. Nos. 3,867,517 and 4,376,1 10. Radiolabeling elements which are useful in imaging include ¹²³I, ¹³¹I, ¹¹¹In, and ^{99m}Tc, for example. Procedures for iodinating antibodies are described by Greenwood et al. (1963) Biochem. J 89:114-123; Marchalonis (1969) Biochem. J. 113:299-305; and Morrison et al. (1971) Immunochemistry 289-297. Procedures for ^{99m}Tc-labeling are described by Rhodes et al. in Burchiel et al. (eds.), Tumor Imaging: The Radioimmunochemical Detection of Cancer, New York: Masson 111-123 (1982) and the references cited therein. Procedures suitable for ¹¹¹In-labeling antibodies are described by Hnatowich et al. (1983) J. Immul. Methods, 65:147-157, Hnatowich et al. (1984) J. Applied Radiation 35:554-557, and Buckley et al. (1984) F.E.B.S. 166:202-204.

In the case of a radiolabeled ligand, the ligand is administered to the patient, is localized to the tumor bearing the alpha₆p with which the ligand reacts, and is detected or “imaged” in vivo using known techniques such as radionuclear scanning using e.g., a gamma camera or emission tomography. See e.g., Bradwell et al. “Developments in Antibody Imaging,” Monoclonal Antibodies for Cancer Detection and Therapy, Baldwin et al., (eds.), pp 65-85 (Academic Press 1985). Alternatively, a positron emission transaxial tomography scanner, such as designated Pet VI located at Brookhaven National Laboratory, can be used where the radiolabel emits positrons (e.g., ¹¹C, ¹⁸F, ¹⁵O, and ¹³N).

MRI Contrast Agents. Magnetic Resonance Imaging (MRI) uses NMR to visualize internal features of living subject, and is useful for prognosis, diagnosis, treatment, and surgery. MRI can be used without radioactive tracer compounds for obvious benefit. Some MRI techniques are summarized in EP-A-0 502 814. Generally, the differences related to relaxation time constants T1 and T2 of water protons in different environments is used to generate an image. However, these differences can be insufficient to provide sharp high-resolution images.

The differences in these relaxation time constants can be enhanced by contrast agents. Examples of such contrast agents include a number of magnetic agents paramagnetic agents (which primarily alter T1) and ferromagnetic or superparamagnetic (which primarily alter T2 response). Chelates (e.g., EDTA, DTPA and NTA chelates) can be used to attach (and reduce toxicity) of some paramagnetic substances (e.g., . Fe⁺³, Mn⁺², Gd⁺³). Other agents can be in the form of particles, e.g., less than 10 μm to about 10 nM in diameter). Particles can have ferromagnetic, antiferromagnetic or superparamagnetic properties. Particles can include, e.g., magnetite (Fe₃O₄), γ-Fe₂O₃, ferrites, and other magnetic mineral compounds of transition elements. Magnetic particles may include: one or more magnetic crystals with and without nonmagnetic material. The nonmagnetic material can include synthetic or natural polymers (such as sepharose, dextran, dextrin, starch and the like

The anti-alpha₆p ligands can also be labeled with an indicating group containing of the NMR-active ¹⁹F atom, or a plurality of such atoms inasmuch as (i) substantially all of naturally-abundant fluorine atoms are the ¹⁹F isotope and, thus, substantially all fluorine-containing compounds are NMR-active; (ii) many chemically active polyfluorinated compounds such as trifluoracetic anhydride are commercially available at relatively low cost, and (iii) many fluorinated compounds have been found medically acceptable for use in humans such as the perfluorinated polyethers utilized to carry oxygen as hemoglobin replacements. After permitting such time for incubation, a whole body MRI is carried out using an apparatus such as one of those described by Pykett (Scientific American, 246:78-88, 1982) to locate and image cancerous tissues.

Also within the scope of the invention are kits comprising the protein ligand that binds to alpha₆p and instructions for diagnostic use, e.g., the use of the anti-alpha₆p ligand (e.g., antibody or antigen-binding fragment thereof, or other polypeptide or peptide) to detect alpha₆p, in vitro, e.g., in a sample, e.g., a biopsy or cells from a patient having a cancer or neoplastic disorder, or in vivo, e.g., by imaging a subject. The kit can further contain a least one additional reagent, such as a label or additional diagnostic agent. For in vivo use the ligand can be formulated as a pharmaceutical composition.

The following invention is further illustrated by the following examples, which should not be construed as further limiting. The contents of all references, pending patent applications and published patents, cited throughout this application are hereby expressly incorporated by reference for all purposes. This application also incorporates by reference the disclosure of the application, Ser. No. ______, entitled “Integrin Protein,” naming Anne Cress as inventor, filed on Mar. 6, 2003, and bearing attorney docket number 10280-067001, in its entirety for all purposes.

### EXAMPLES

Evaluating a Sample for a Disease or Disorder

Various disease states involving epithelial cells have been associated with alterations in alpha6 integrin-containing heterodimers. Mice completely lacking the alpha6 integrin will develop to birth but die shortly thereafter because of severe blistering of the skin and other epithelia (Georges-Labouesse et al., Nat. Genet. 13:370-373, 1996). Alterations in the alpha6 integrin and/or a deficiency of its pairing subunit, beta4 integrin, are associated with pyloric atresia-junctional epidermolysis bullosa, a human blistering disease of the epithelia (Brown et al., J. Invest. Dermatol. 107:384-391, 1996; Shimizu et al., Arch. Dermatol. 132:919-925, 1996; Vidal et al., Nat. Genet. 10:229-234, 1995; Pulkkinen et al., Lab. Invest. 76:823-833, 1997; Niessen et al., J. Cell Sci. 109:1695-1706, 1996; Gil et al., J. Invest. Dermatol. 103:31S-38S, 1994).

Investigation of a human epithelial cancer indicated a deficiency of the alpha6beta4 heterodimer pairing during prostate tumor progression (Cress et al., Cancer Metastasis Rev. 14:219-228, 1995; Nagle et al., Am. J. Pathol. 146:1498-1507, 1995) and a persistent expression of the alpha6beta1 integrin (Knox et al., Am. J. Pathol. 145:167-174, 1994). Other studies have revealed the persistent nonpolarized expression of the alpha6 integrin during human tumor progression in cancers arising within the breast (Friedrichs et al, Cancer Res. 55:901-906, 1995), kidney (Droz et al., Lab. Invest. 71:710-718, 1994), endometrium (Lessey et al., Am. J. Pathol. 146:717-726, 1995), and pancreas (Weinel et al., Int. J. Cancer 52:827-833, 1992; Weinel et al., Gastroenterology 108:523-532, 1995), in addition to micrometastases from solid epithelial tumors (Putz et al., Cancer Res. 59:241-248, 1999). Immunoprecipitation of alpha6 integrin from human prostate cancer cells using alpha6-specific monoclonal antibodies retrieved not only the expected alpha1 and beta4 subunits but also a predominant protein with an apparent molecular mass of 70 kDa (Witkowski et al., J. Cancer Res. Clin. Oncol. 119:637-644, 1993; Rabinovitz et al., Clin. Exp. Metastasis 13:481-491, 1995). The results presented below show that the protein is a novel and smaller form of the alpha6 integrin that is capable of pairing with either the alpha1 or beta4 integrin subunit. The integrin is referred to herein as an N-terminally truncated alpha6 integrin, and is called alpha6p for the latin word parvus, meaning small.

### Example 1

Materials and Methods

Cell Lines. All human cell lines were incubated at 37° C. in a humidified atmosphere of 95% air and 5% CO₂. Cell lines DU145H, HaCaT, and PC3-N were grown in Iscove's modified Dulbecco's medium (Invitrogen Corporation, Carlsbad, Calif.; formerly Life Technologies, Inc.) plus 10% fetal bovine serum. Cell lines MCF-7, PC3-ATCC, LnCap, and H69 were grown in Dulbecco's modified Eagle's medium (Invitrogen Corporation, Carlsbad, Calif.; formerly Life Technologies, Inc.) plus 10% fetal bovine serum. SW480 cells were grown in super medium (Dulbecco's modified Eagle's medium plus 5% nonessential amino acids, 5% L-glutamine, 5% sodium pyruvate, 10% fetal bovine serum). Normal prostate cells, PrEC, were grown in PrEGM bullet kit medium (Clonetics, San Diego, Calif.). The following cell lines were obtained from the American Type Culture Collection (Manassas, Va.): MCF-7 (human breast tumor), PC3 (human prostate tumor), LnCap (prostate carcinoma cell line), H69 (human lung carcinoma), and SW480 (human colon carcinoma). The DU145H cells were isolated as described previously (Rabinovitz et al. (1995) Clin. Exp. Metastasis 13:481-491) and contain only the alpha6A splice variant (Cress et al. (1995) Cancer Metastasis Rev. 14:219-228). The PC3-N cells are a variant of PC3 prostate carcinoma cell line (Tran et al. (1999) Am. J. Pathol. 155:787-798). The HaCaT cells (normal immortalized keratinocyte cell line) (Breitkreutz et al. (1998) Eur J. Cell Biol. 75:273-286) were obtained from Dr. Norbert E. Fusenig (German Cancer Research Center, University of Heidelberg, Heidelberg, Germany). PrEC (a normal prostate cell line) was obtained from Clonetics. The calcium-induced terminal differentiation assay, cell culture techniques, and preparation of calcium medium used for mouse 291, 03C, and 03R cells have been described previously (Kulesz-Martin et al. (1980) Carcinogenesis 1:995-1006; Hennings et al. (1980) Curr. Probl. Dermatol. 10:3-25). Cells were maintained in 0.04 mM calcium (low calcium) and switched to medium with 0.14 mM calcium (medium calcium) or 1.4 mM calcium (high calcium) by 60% confluency. After 24 h treatment, cells were collected in phosphate-buffered saline, centrifuged, frozen in a dry ice bath, and kept at 80° C. in a freezer until used.

Antibodies. Anti-alpha6 integrin antibodies include and were obtained as follows. GoH3, a rat IgG2a, was from Accurate Chemicals (Westbury, N.Y.) (Sonnenberg et al. (1987) J. Biol. Chem. 262:10376-10383); J1B5, a rat monoclonal antibody, was obtained from Dr. Caroline Damsky (University of California, San Francisco, Calif.) (Damsky et al. (1994) Development 120:3657-3666); 4F10, a mouse IgG2b, was from Chemicon (Temcula, Calif.) (Sonnenberg et al. (1987) J. Biol. Chem. 262:10376-1038); BQ16, a mouse IgG1 that recognizes an extracellular epitope of the alpha6 integrin, was obtained from Dr. Monica Leibert (Department of Urology, University of Texas, M. D. Anderson Cancer Center, Houston, Tex.) (Liebert et al. (1993) Hybridoma 12:67-80); 4E9G8, a mouse IgG1 that is specific for the unphosphorylated alpha6A cytoplasmic tail, was from Immunotech (Marseille, France) (Sonnenberg et al. (1988) Nature 336:487-489; Hemler et al. (1988) J. Biol. Chem. 263:7660-7665); AA6A, a rabbit polyclonal antibody that was raised and purified by Bethyl Laboratories Inc. (Montgomery, Tex.) specific for the last 16 amino acids (CIHAQPSDKERLTSDA; SEQ ID NO:3) of the human alpha6A sequence (Tamura et al. (1990) J. Cell Biol. 111:1593-1604) as done previously (Cooper et al. (1991) J. Cell Biol. 115:843-850), and A33, a rabbit polyclonal antibody that was raised against amino acids 1-500 of the alpha6 integrin (Sterk et al. (2000) J. Cell Biol. 149:969-982), were generous gifts from Dr. Arnoud Sonnenberg (The Netherlands Cancer Institute). Anti-alpha4 integrin antibodies were obtained as follows. 3E1, a mouse ascites IgG1, was from Invitrogen Corporation (Carlsbad, Calif.; formerly Life Technologies, Inc.) (Hessle et al. (1984) Differentiation 26:49-54); 439.9b, a rat IgG2bK, was from Pharmingen (San Diego, Calif.) (Falcioni et al. (1988) Cancer Res. 48:816-821); ASC-3, a mouse IgG1K, was from Chemicon (Temecula, Calif.) (Pattaramalai et al. (1996) Exp. Cell Res. 222:281-290); and A9, a mouse IgG2a, was from Ancell (Bayport, Minn.) (Van Waes et al. (1991) Cancer Res. 51:2395-2402). Other anti-integrin antibodies include anti-alpha5 integrin antibody P1D6, a mouse IgG3 (Invitrogen Corporation, Carlsbad, Calif.; formerly Life Technologies, Inc.) (Wayner et al. (1988) J. Cell Biol. 107:1881-1891), and anti-alpha1 integrin P4C1O, a mouse ascites IgG1 (Invitrogen Corporation, Carlsbad, Calif.; formerly Life Technologies, Inc.) (Carter et al. (1990) J. Cell Biol. 110:1387-1404).

Surface Biotinylation of Cell Lines. Previous published protocols (Isberg and Leong (1990) Cell 60:861-871; Einheber et al. (1993) J. Cell Biol. 123:1223-1236) were slightly modified. Briefly, cells were grown to confluency in 100-mm tissue culture dishes and washed three times with HEPES buffer (20 mM HEPES, 130 mM NaCl, 5 mM KCl, 0.8 mM MgCl₂, 1.0 mM CaCl₂, pH 7.45). The cells were then incubated with 2 ml of HEPES buffer supplemented with sulfosuccinimidyl hexanoate-conjugated biotin (500 μg/ml; Pierce), which is impermeable to cell membranes (Staros (1982) Biochemistry 21:3950-3), to label cell surface proteins for 30 min at 4° C. The cells were washed three times and lysed in cold radioimmune precipitation buffer plus protease inhibitors (phenylmethylsulfonyl fluoride, 2 mM; leupeptin and aprotinin, 1 μg/ml). The lysate was briefly sonicated on ice before centrifugation at 10,000 rpm for 10 min, and the supernatant was collected for immunoprecipitations.

Immunoprecipitations. For immunoprecipitations, 200 μg of total protein lysate was used for each reaction and incubated with 35 μl of protein G-Sepharose and 1 μg of antibody. The final volume of the lysate was adjusted to 500 μl with radioimmune precipitation buffer (150 mM NaCl, 50 mM Tris, 5 mM EDTA, 1% (v/v) Triton X-100, 1% (w/v) deoxycholate, 0.1% (w/v) SDS, pH 7.5). The mixture was rotated for 18 h at 4° C. After incubation, complexes were washed three times with cold radioimmune precipitation buffer and eluted in 2×non-reducing sample buffer. Samples were boiled for 5 min prior to loading onto a 7.5% polyacrylamide gel for analysis. The proteins resolved in the gel were electrotransferred to Millipore (Billerica, Me.) Immobilon-P polyvinylidene fluoride membrane, incubated with either peroxidase-conjugated streptavidin or Western blotting antibodies plus secondary antibody conjugated to horseradish peroxidase, and visualized by chemiluminescence (ECL Western blotting detection system; Amersham Pharmacia Biotech, Piscataway, N.J.).

Two-dimensional Nonreduced/Reduced Gel Electrophoreses. Nonreduced/reduced two-dimensional electrophoresis was done as described (Parker et al. (1993) J. Biol. Chem. 268:7028-7035). The samples were incubated in 0.625 M Tris-HCl, pH 6.8, 10% glycerol, 10% SDS, and applied to SDS-polyacrylamide gel electrophoresis (7.5% acrylamide) without reduction. The excised lanes were incubated in reducing sample buffer for 15 min and horizontally loaded at the top of a second dimension slab gel (also 7.5% acrylamide). The proteins were electrotransferred to polyvinylidene fluoride membrane (Millipore, Billerica, Me.), incubated with either peroxidase-conjugated streptavidin or Western blotting primary antibodies followed by secondary antibody conjugated to horseradish peroxidase, and visualized by chemiluminescence (ECL Western blotting detection system; Amersham Pharmacia Biotech, Piscataway, N.J.).

Amino Acid Sequencing by Matrix-assisted Laser Desorption Ionization Mass Spectrometry and Liquid Chromatography-Tandem Mass Spectrometry. Amino acid sequencing of alpha6p was performed using two different analytical core services. For analytical core service at Deutsches Krebsforschungszentrum (Heidelberg, Germany), the alpha6p protein was immunoprecipitated using J1B5, and the proteins were separated by SDS-polyacrylamide gel electrophoresis (7.5%, 3 mm). After staining with Coomassie Blue, the alpha6p bands were excised, cut into small pieces (1×1 mm), washed, dehydrated (twice for 30 min with H₂O, twice for 15 min with 50% acetonitrile, and once for 15 min with acetonitrile), and incubated with 0.5 μg of trypsin in 20 μl of digest buffer (40 mM NH₄HCO₃, pH 8.0) at 37° C. for 16 h. The supernatant was subsequently analyzed by matrix-assisted laser desorption ionization (MALDI) mass spectrometry (Deutsches Krebsforschungszentrum) using thin film preparation technique. Aliquots of 0.3 μl of a nitrocellulose containing saturated solution of alpha-cyano-4-hydroxycinnamic acid in acetone were deposited onto individual spots on the target. Subsequently, 0.8 μl of 10% formic acid and 0.4 μl of the digest sample were loaded on top of the thin film spots and allowed to dry slowly at ambient temperature. To remove salts from the digestion buffer, the spots were washed with 5% formic acid and with H₂O. Sequence analysis was performed on a Procise 494 protein sequencer using a standard program supplied by Applied Biosystems (Foster City, Calif.). The FastA database searching program of Pearson and Lipman (Pearson and Lipman (1988) Proc. Natl. Acad. Sci. U.S.A. 85:2444-2448) was used for database searching.

For sequence analysis at the Proteomics Core of the Arizona Cancer Center and Southwest Environmental Health Sciences Center of the University of Arizona, the alpha6p protein was immunoprecipitated using J1B5 antibody, and proteins were separated by SDS-polyacrylamide gel electrophoresis (7.5%, 3 mm). After staining with Coomasie Blue, the alpha6p bands were excised, cut into small pieces (1×1 mm), and subjected to in-gel digestion using trypsin as described previously (Shevchenko et al. (1996) Anal. Chem. 68:850-858). The extracted peptides following digestion were analyzed by liquid chromatography-tandem mass spectrometry using a quadrupole ion trap Finnigan LCQ classic mass spectrometer equipped with a quaternary pump P4000 HPLC and a Finnigan electrospray ionization source (ThermoFinnigan, San Jose, Calif.). The peptides were eluted from a reverse-phase C18 micro-column (Vydac 250×1 mm, Hesperia, Calif.) with a gradient of 3-95% acetonitrile in 0.5% formic acid and 0.01% trifluoroacetic acid over 150 min at a flow rate of 15 μl/min. Tandem mass spectrometry spectra of the peptides were analyzed with the SEQUEST program (Turbo Sequest) to assign peptide sequence to the spectra (Eng et al. (1994) J. Am. Soc. Mass Spectrom Vol. 5). SEQUEST analyses were performed against the nonredundant database.

RT-PCR Analysis. Total cellular RNA was isolated by guanidium isothiocyanate cell lysis and cesium chloride purification (Chirgwin et al. (1979) Biochemistry 18:5294-5299). RNA was quantitated from spectrophotometric absorbance measurements at 260 nm. First strand cDNA was synthesized in a 30 μl reaction comprised of 1×PCR buffer (10 mM Tris, pH 8.3; 50 mM KCl; 1.5 mM MgCl₂); 1 mM each dATP, dCTP, dGTP, and dTTP; 100 pmol random hexamer; 20 units RNAsin; 200 units SuperScript reverse transcriptase II (Invitrogen Corporation, Carlsbad, Calif.; formerly Life Technologies, Inc.), and 3 μg of total cellular RNA incubated at 42° C. for 60 min. The reaction was terminated by incubation at 99° C. for 10 min. Integrin alpha6-specific PCR was performed by adding 80 μl of amplification reaction buffer (1×PCR buffer, 25 pmol of integrin alpha6-specific primers, and 2.5 units of Taq DNA polymerase) to the cDNA reaction, followed by incubation at 94° C. for 5 min and then 40 cycles of 94° C. for 1 min, 60° C. for 3 min, and 72° C. for10 min, with final extension at 72° C. for 5 min and a quick chill to 4° C. The PCR primers were derived from the integrin alpha6 cDNA sequence reported by Tamura et al. (J. Cell Biol. 111:1593-1604, 1990) (GenBank accession number X53586); the upstream primer sequence was from nucleotides 160 to 179, and the downstream primer was from nucleotides 3404 to 3423. The PCR product identity was confirmed by diagnostic restriction enzyme digests and size separation of the products through a 1×TBE, 1.5% agarose gel. The products were visualized by ethidium bromide staining and UV fluorescence.

### Example 2

DU145H Cells Contained a Smaller Form of the Alpha6 Integrin

Previous studies showed that the anti-alpha6 antibody GoH3 can immunoprecipitate a surface-biotinylated 70-kDa (non-reduced) protein from DU145H cells in addition to the expected 185-, 140-, and 120-kDa (non-reduced) proteins corresponding to the alpha4, alpha6, and alpha1 integrins, respectively (Witkowski et al. (1993) J. Cancer Res. Clin. Oncol. 119:637-644; Rabinovitz et al. (1995) Clin. Exp. Metastasis 13:481-491). In DU145H cells, which only contain the alpha6A splice variant of alpha6 integrin (Cress et al. (1995) Cancer Metastasis Rev. 14:219-228), this 70-kDa variant was the predominant form of the alpha6 integrin found on the cell surface.

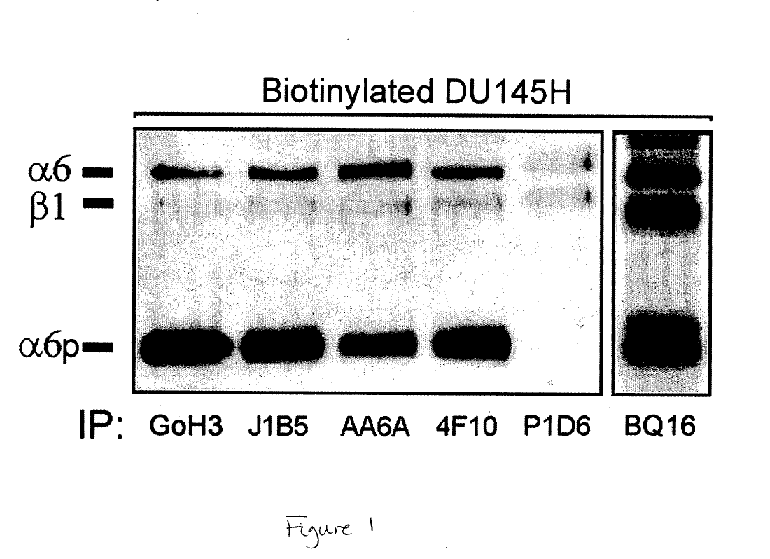

Five different anti-alpha6 antibodies immunoprecipitated alpha6 and its smaller variant, alpha6p, from surface-biotinylated DU145H cells (FIG. 1). Four of the antibodies used were specific for extracellular epitopes of the full-length alpha6A integrin (GoH3, J1B5, 4F10, and BQ16), and one was specific for the cytoplasmic tail of the alpha6A light chain (AA6A). The integrin alpha6p was not found to co-immunoprecipitate upon incubation with an anti-alpha3 antibody, P1B5 (data not shown), or an anti-alpha5 antibody, P1D6.

### Example 3

The Alpha6p Variant Contained a Light Chain That was Identical to That Found in Alpha6 Integrin

The full-length alpha6 integrin consists of two disulfide-linked chains, a heavy chain (110-kDa) and a cytoplasmic light chain (30-kDa) that are observed upon reduction of the protein samples and analysis by SDS-polyacrylamide gel electrophoresis. Western blot analysis (FIG. 1) indicated that an anti-alpha6 integrin antibody specific for the cytoplasmic tail of alpha6A (antibody AA6A) recognized alpha6p and suggested that the light chain from the alpha6p variant might be similar to that in the full-length alpha6 integrin.

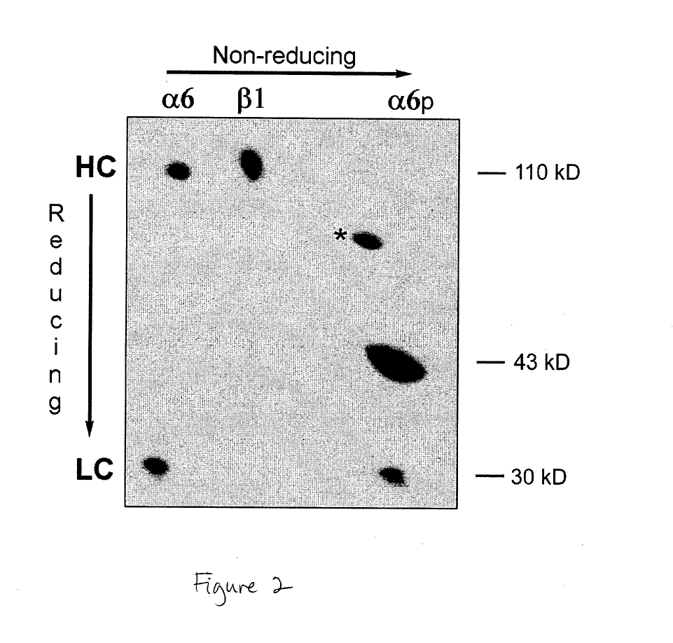

To test this hypothesis, DU145H cells were surface-biotinylated, and then immunoprecipitations were performed using the anti-alpha6 integrin antibody, GoH3. The immunoprecipitated protein was then analyzed using two-dimensional non-reducing/reducing gel electrophoresis (FIG. 2). The sample was electrophoresed under non-reducing conditions in the first dimension and then under reducing conditions for the second dimension. The 160-kDa band (non-reduced) corresponding to the full-length alpha6 integrin contained a heavy (110-kDa) and light (30-kDa) chain, as described previously (Hogervorst et al. (1991) Eur. J. Biochem. 199:425-433). The reduced alpha1 integrin was identified at 120-kDa. The alpha6p integrin split into a heavy fragment (43-kDa) and a light chain (30-kDa). These results indicated that the alpha6p integrin contained the same 30-kD light chain as the full-length alpha6A integrin but the heavy chains were significantly different.

### Example 4

The Alpha6p Variant Associated with Beta1 and Beta4 Integrins

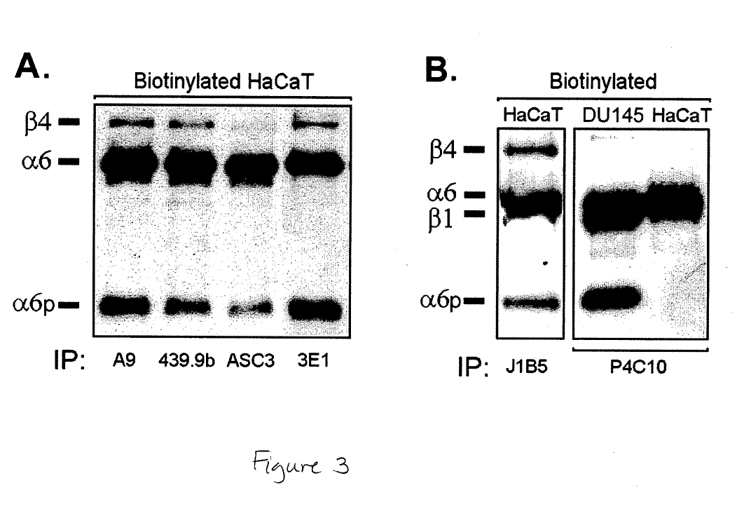

The alpha6 integrin is known to associate with either the beta1 or beta4 integrin subunit (Sonnenberg et al. (1990) J. Cell Sci. 96:207-217). Alpha6p was tested to determine whether it would co-immunoprecipitate with the beta4 integrin (FIG. 3A). Human HaCaT cells were chosen for this experiment because of their abundance of beta4 integrin (Witkowski et al. (1993) J. Cancer Res. Clin. Oncol. 119:637-644). The cells were surface-biotinylated and subjected to immunoprecipitation with different anti-beta4 integrin antibodies. The alpha6p variant co-immunoprecipitated upon incubation with four different anti-beta4 integrin antibodies: A9, 439.9b, ASC3, and 3E1. Of particular interest was the immunoprecipitation of alpha6p with the anti-beta4 integrin antibody, A9, whose epitope is present when alpha6 is coupled to beta4 integrin (Van Waes et al. (1991) Cancer Res. 51:2395-2402).

Next, the anti-beta1 integrin monoclonal antibody, P4C10, was tested for its ability to coimmunoprecipitate the novel 70-kDa (non-reduced) protein (FIG. 3B). HaCaT cells were surface-biotinylated and immunoprecipitated with anti-alpha6 integrin antibody, J1B5, and used as a standard. Both DU145 and HaCaT cells were surface-biotinylated and subjected to immunoprecipitation using P4C10. Interestingly, the 70-kDa (non-reduced) alpha6p variant co-immunoprecipitated with the beta1 integrin in DU145 cells but not in HaCaT cells. The results indicated that the novel alpha6p variant paired with either the beta4 or beta1 integrin subunits. Although the beta1 integrin was readily present in HaCaT cells, the alpha6p integrin did not co-immunoprecipitate with the anti-beta1 integrin antibody, P4C10.

### Example 5

The Alpha6p Integrin was Recognized by Light Chain-Specific Anti-Alpha6A Monoclonal Antibodies

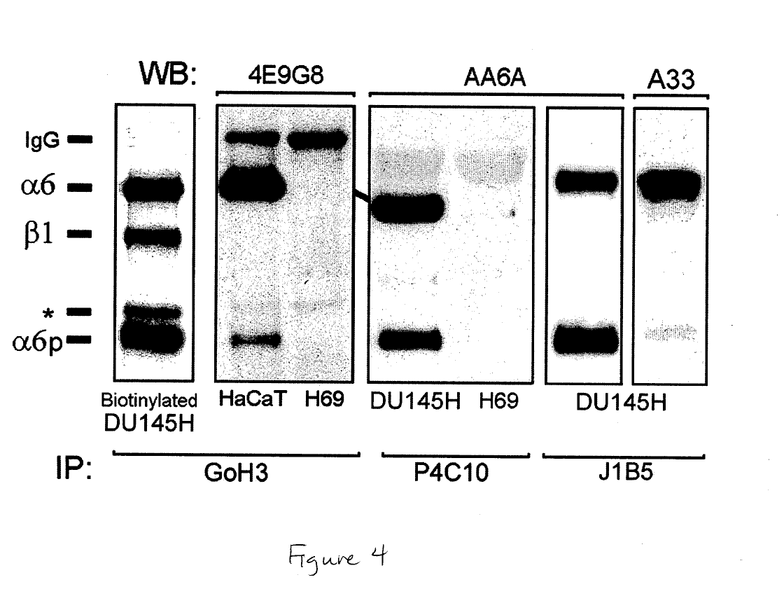

The data presented in (FIG. 2) indicated that alpha6p contained a light chain identical to that contained in the full-length alpha6 integrin. An experiment was performed to determine whether an anti-alpha6 integrin antibody could recognize the novel 70-kDa (non-reduced) protein by Western blot. DU145H cells were surface-biotinylated and immunoprecipitated with GoH3 antibodies for a standard to compare with a Western blot (FIG. 4). DU145H, HaCaT, and H69 cells were lysed and immunoprecipitated with either anti-alpha6 integrin antibodies GoH3 or J1B5 or anti-beta1 integrin monoclonal antibody, P4C10. A 70-kDa band that co-migrated with the biotinylated standard was recognized in HaCaT and DU145H cells by Western blot analysis using two different anti-alpha6A antibodies, AA6A and 4E9G8, which recognize the cytoplasmic domain of the alpha6A integrin. Additionally the alpha6 integrin, but not the alpha6p variant, was detected by Western blot analysis using A33, which is specific for the N-terminus of the alpha6 integrin. A lung carcinoma cell line, H69, is a cell line that does not contain alpha6 integrin and was not found to express alpha6p.

### Example 6

The Alpha6p Variant Was Present in Several Different Epithelial Cancer Cell Lines

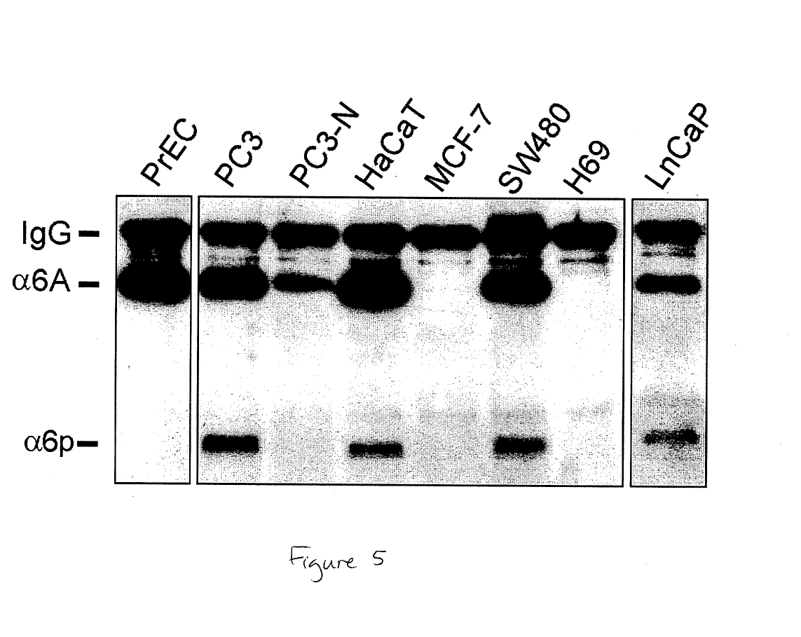

A variety of tumor and normal cell lines was examined for the presence of the alpha6p variant. The presence of alpha6 and alpha6p was initially analyzed by using whole cell lysates (20 μg of total protein) followed by Western blot analysis (data not shown). The results were tabulated and confirmed by immunoprecipitation with anti-alpha6 antibody GoH3 followed by Western blot analysis using anti-alpha6A antibody, AA6A (FIG. 5). The alpha6p variant was present in several prostate cancer cell lines (DU145H, PC3, and LnCaP) and a colon cancer cell line (SW480). Additionally, alpha6p was present in a normal, immortalized keratinocyte cell line, HaCaT. The alpha6p variant was not found in several cell lines including normal prostate cells, PrEC; a variant of the prostate cell line PC3, called PC3-N (Tran et al. (1999) Am. J. Pathol. 155:787-798); a breast carcinoma cell line, MCF-7; and a lung carcinoma cell line, H69. Interestingly, the alpha6p variant was only observed in cells that expressed the full-length alpha6 integrin. The alpha6p variant was not present in alpha6-negative cell lines. Two epithelial cell lines, one normal cell line (PrEC) and one cancer cell line (PC3-N), expressed the full-length alpha6 integrin but not the alpha6p variant.

### Example 7

The Alpha6p Variant Contained Several Amino Acid Fragments Identical to the Alpha6 Integrin

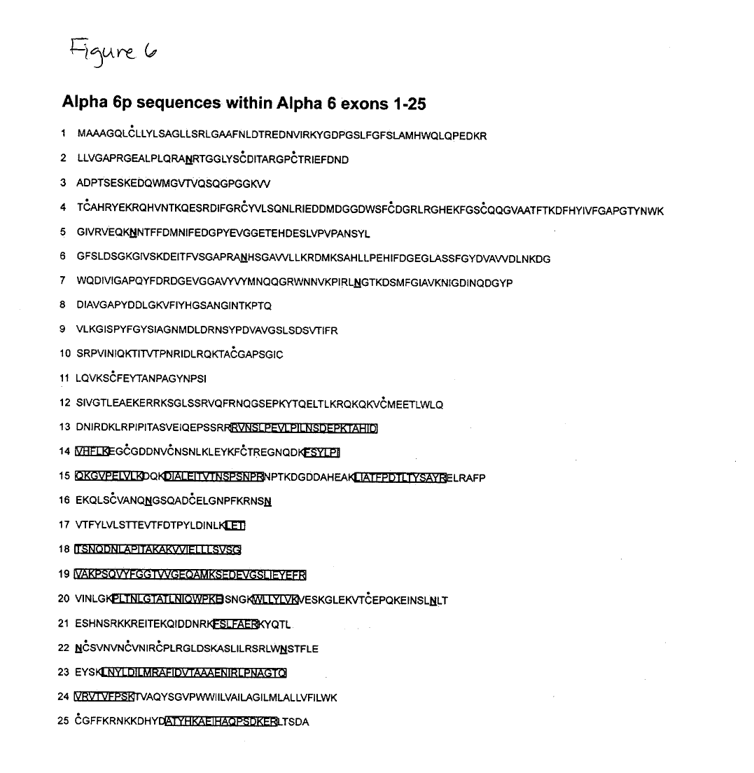

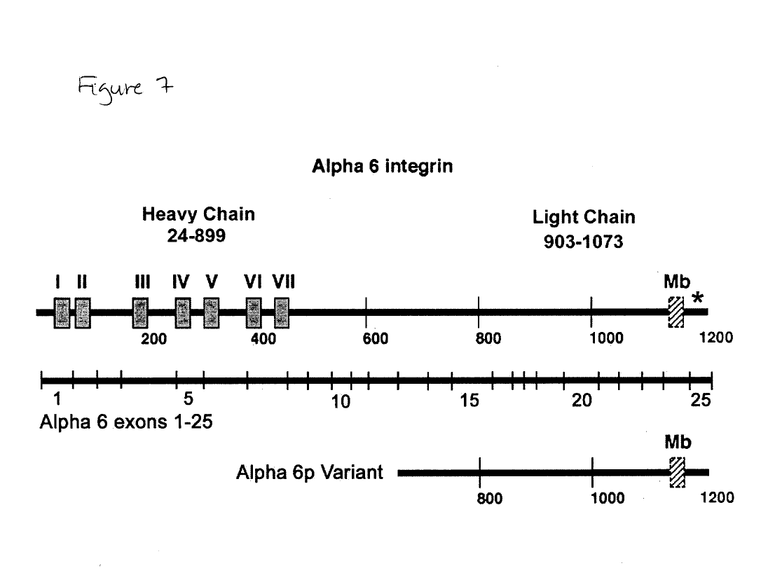

To determine more precisely the identity of the alpha6p variant, the protein was isolated and sequenced. To isolate the protein, alpha6p was immunoprecipitated with J1B5 antibody and electrophoresed. The protein gel was stained with Coomassie Blue, and then the 70-kDa protein was excised and digested with trypsin. Protein sequences were obtained using either MALDI mass spectrometry (Deutsches Krebsforschungszentrum) or liquid chromatography-tandem mass spectrometry (Proteomics Core of the Arizona Cancer Center and Southwest Environmental Health Sciences Center, University of Arizona) (FIG. 6). Ten noncontinuous amino acid fragments within the alpha6p variant were identified that corresponded exactly to predicted trypsin fragments located on exons 13-25 of the published alpha6 integrin sequence (Tamura et al. (1990) J. Cell Biol. 111:1593-1604). The sequencing data confirmed that both the heavy and light chains of the alpha6p variant contained identical portions of the full-length alpha6 integrin (FIG. 7).

### Example 8

The Alpha6p Variant Half-life was Three Times Longer than Alpha6

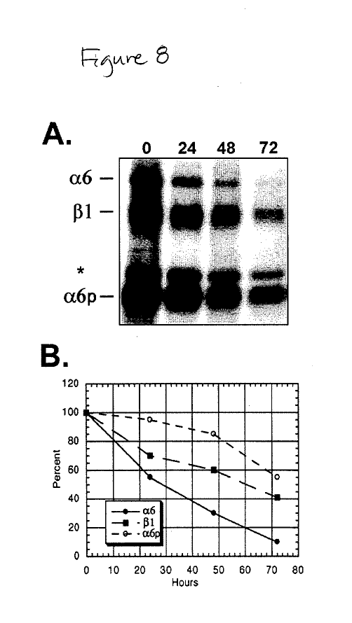

To determine if the novel alpha6p variant was a degradation product of the alpha6 integrin that would be rapidly cleared from the surface, the surface half-life of both integrins was examined using a biotinylation strategy. The surface proteins of DU145H cells were biotinylated for 1 h, washed, and placed back in the incubator with medium. After 24, 48, or 72 h, the integrins were immunoprecipitated using the GoH3 antibody and analyzed under non-reducing conditions (FIG. 8, panel A). The data indicated that the alpha6p form remained on the surface of the DU145H cells with a half-life of ˜72 h, or almost 3 times longer than that of the full-length alpha6 integrin (FIG. 8, panel B). The abundance of alpha6p was not influenced by exogenous protease inhibitors (BB94, leupeptin, aprotinin, 30% fetal bovine serum, ecotin), exogenous proteases (kallikrein), or activators of integrin function (12-O-tetradecanoylphorbol-13-acetate, 20 mM CaCl₂) (data not shown).

### Example 9

RT-PCR Analysis of the Alpha6 Coding Region Revealed a Single Product

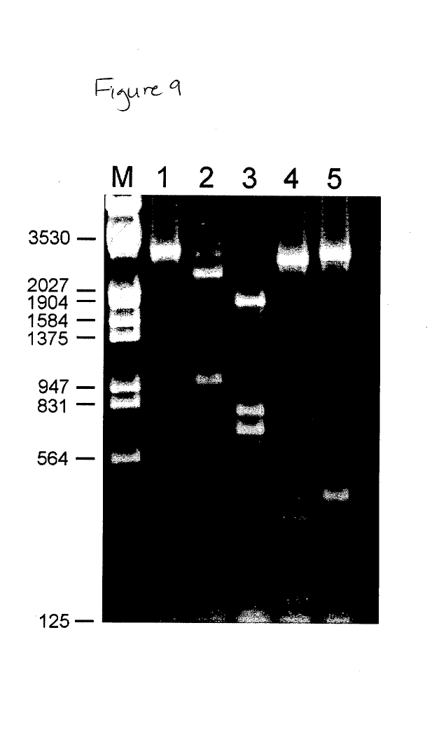

RT-PCR was used to determine whether splice variants of the integrin alpha6 mRNA could explain the production of the smaller integrin protein. Three micrograms of total cellular RNA from DU145H cells were reverse-transcribed into first strand cDNA and then PCR amplified with primers that bracketed most of the integrin alpha6 protein-coding region (all but the first four codons were amplified using these primers). The results of this experiment are shown in FIG. 9. A single PCR product consistent with a full-length RT-PCR product of 3263 bp was detected.

To confirm the identity of the integrin alpha6 PCR product, diagnostic restriction enzyme digests were performed. Analysis of the integrin alpha6 sequence (Tamura et al. (1990) J. Cell Biol. 111:1593-1604) revealed the presence of one EcoN I site (producing fragments of ˜960 and 2300 bp), four Sma I restriction sites (producing fragments of 105, 150, 350, and 2650 bp), four EcoR I sites (producing fragments of 30, 680, 730, and 1780 bp), and one Xho I site (producing fragments of 420 and 2840 bp). Aliquots of the integrin alpha6 PCR product were digested with each of these restriction enzymes, and the results of this experiment are shown in FIG. 9. Each restriction digest product produced the restriction fragments expected from the integrin alpha6 PCR product (the 30 bp EcoR I fragment and the 105 bp Sma I fragment could not be visualized on the gel shown in FIG. 9). Based on these results, it appears unlikely that the alpha6p variant is the result of the splicing out of exons in the known coding region.

### Example 10

Calcium-Induced Normal Keratinocyte Differentiation Increased Alpha6p Integrin Protein Levels

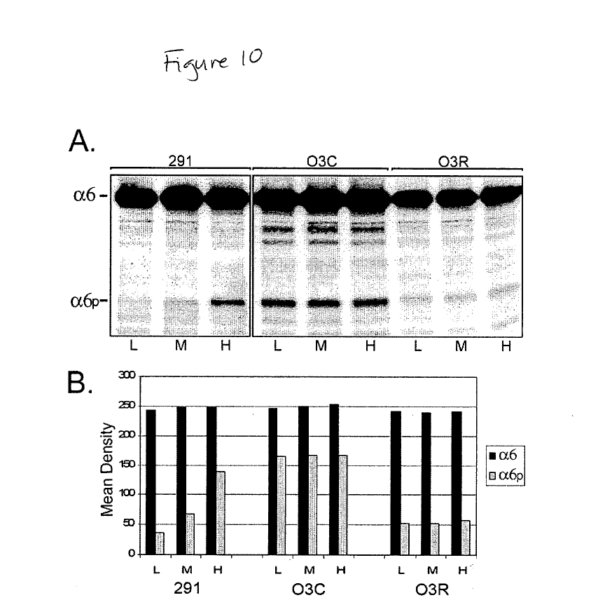

Mouse 291 normal keratinocyte terminal differentiation can be induced by calcium. O3C and O3R cells were derived from normal 291 mouse cell strains and are immortalized, nontumorigenic, and tumorigenic, respectively (Kulesz-Martin et al. (1988) Carcinogenesis 9:171-174). Both cell strains are resistant to calcium-induced terminal differentiation. The presence of alpha6 and alpha6p integrins in normal 291 mouse keratinocytes was determined using whole cell lysates followed by Western blot analysis using anti-alpha6 integrin antibody, AA6A (FIG. 10A). The results for 291 cells were confirmed by immunoprecipitation with anti-alpha6 integrin antibody, GoH3 (data not shown). The alpha6 and alpha6p integrin protein bands were quantitated using Scion Image (Cress (2000) BioTechniques 29:776-781), and the results were graphed (FIG. 10B). Calcium-induced terminal differentiation increased alpha6p integrin protein levels 3-fold in a dose-dependent manner in 291 nontransformed mouse keratinocytes. The differing steady-state levels of alpha6p in proliferating O3C and O3R tumor cells under the same culture conditions suggested that the alpha6p integrin variant was responsive to terminal differentiation and not to calcium itself. Interestingly, alpha6p integrin levels were decreased in poorly differentiated squamous cell carcinoma O3R cells relative to initiated cell O3C precursors and terminally differentiated 291 keratinocytes.

Other embodiments are within the following claims.

We claim:

1. A method of screening for a ligand that has an ability to bind to an N-terminal truncated form of an alpha6 integrin, the method comprising:
providing a library of test ligands,
contacting members of the library with an N-terminal truncated form of an alpha6 integrin, and
identifying one or more members that bind to the N-terminal truncated form.

2. The method of claim 1, further comprising
contacting the one or more identified members to a full-length, mature alpha6 integrin and
selecting one or more members that bind to the N-terminal truncated alpha6 integrin with a higher affinity than to the full-length, mature alpha6 integrin.

3. The method of claim 1, wherein the library comprises a plurality of display library members.

4. The method of claim 1, wherein the N-terminal truncated form of the integrin is a naturally-occurring proteolytic product of the full-length form.

5. The method of claim 1, wherein the N-terminal truncated form is attached to the surface of a cell.

6. The method of claim 3, wherein the library comprises a plurality of members that each include an immunoglobulin variable domain.

7. The method of claim 3, wherein each display library member of the plurality is a phage particle that comprises a test ligand physically associated with a bacteriophage coat protein.

8. The method of claim 1, wherein the N-terminal region of the N-terminal truncated form is formed by urokinase cleavage of the full-length, mature form of the integrin.

9. An isolated protein ligand that binds a naturally occurring, truncated form of an integrin, and
has a higher binding affinity for the naturally occurring, truncated form of the integrin than for the full-length, mature form of the integrin.

10. The ligand of claim 9, wherein the integrin is an alpha6 integrin, and the naturally occurring truncated form of the alpha6 integrin is an alpha6p integrin.

11. The ligand of claim 10, wherein the binding affinity (Ka) of the ligand for the naturally occurring truncated form of the integrin is at least 10 fold higher than the binding affinity for the mature, full-length form of the integrin.

12. The ligand of claim 10, wherein the naturally occurring truncated form of the integrin is a proteolytic product of the mature, full-length form.

13. The ligand of claim 10, wherein the ligand comprises a plurality of polypeptide chains.

14. The ligand of claim 10, wherein the ligand comprises an immunoglobulin variable domain.

15. The ligand of claim 13, wherein the ligand is an antibody.

16. The ligand of claim 10, wherein the ligand binds to the N-terminus of alpha6p.

17. The ligand of claim 10, wherein the ligand impairs an interaction between alpha6p and a tetraspanin.

18. The ligand of claim 10, wherein the ligand binds to a novel conformational epitope present in the N-terminal truncated form of the integrin and absent in the full-length form of the integrin.

19. An isolated protein ligand that binds to an alpha6 integrin and inhibits interaction between alpha6 integrin and one or more alpha6 interacting proteins selected from the group consisting of: uPAR, urokinase, and a tetraspanin.

20. A method of identifying a ligand that binds specifically to a naturally occurring truncated form of an alpha6 integrin, the method comprising:
(a) providing a test ligand that is encoded by a nucleic acid that comprises a synthetic sequence;
(b) contacting the test ligand with a naturally occurring truncated form of an alpha6 integrin; and
(c) identifying the test ligand as a ligand that binds specifically to the naturally occurring truncated form of an alpha6 integrin if the test ligand binds to the naturally occurring truncated form of an alpha6 integrin but does not substantially bind to a full-length form of the alpha6 integrin.

21. The method of claim 20, wherein the test ligand is selected from a display library.

22. The method of claim 26, wherein the truncated form of the alpha6 integrin is alpha6p.

23. The method of claim 20 wherein the test ligand does not include an immunoglobulin domain.

24. A method of detecting alpha6p in a sample, the method comprising:
contacting the sample with the ligand of claim 9, and
evaluating interaction between the ligand and the sample, thereby detecting alpha6p in the sample.

25. A method of treating a subject, the method comprising:
identifying a subject in need of a protein ligand that has a higher binding affinity for an alpha6p integrin than for an alpha6 integrin, and
administering the ligand to the subject, thereby treating the subject.

26. The method of claim 25 wherein the subject has or is at risk for having a proliferative disorder.

27. The method of claim 26 wherein the subject has a metastatic cancer.

28. The method of claim 25, wherein the ligand is an antibody.

29. The method of claim 25, wherein the ligand comprises a cytotoxin.

30. The method of claim 28, wherein the antibody comprises an Fc region.

31. The method of claim 26, wherein the cancer is an epithelial, prostate, colon, breast, lung, kidney, or pancreatic cancer.

32. The method of claim 25, wherein the subject has or is at risk for having an epithelial disorder.

33. The method of claim 32, wherein the epithelial disorder is an epidermolysis bullosa.

34. The method of claim 30, wherein the ligand reduces cell migration in the subject.

35. The method of claim 25, wherein the subject has or is at risk for having a bleeding disorder or an endothelial cell disorder.

36. A method of evaluating a subject, comprising
providing a biological sample from a subject;
testing for an interaction between a ligand and a naturally occurring truncated form of an alpha6 integrin in the biological sample, wherein the naturally occurring truncated form is an alpha6p integrin, and
correlating an interaction between the ligand and the naturally occurring truncated alpha6 integrin with an increased risk for a disease or disorder, thereby evaluating the subject.

37. The method of claim 36, wherein the disorder is a proliferative or epithelial disorder.

38. The method of claim 36, wherein the cancer is a metastatic cancer.

39. The method of claim 36, wherein the epithelial disorder is an epidermolysis bullosa.

40. The method of claim 36, wherein the biological sample is a biopsy or a blood sample.

41. An isolated nucleic acid molecule comprising a sequence that encodes the protein ligand of claim 9.

42. A host cell that contains the nucleic acid molecule of claim 41.

43. A method of evaluating a subject, the method comprising:
administering the ligand of claim 9 to the subject, wherein the ligand contains a detectable label, and
imaging the subject to detect localization of the ligand within the subject.

44. An isolated polypeptide comprising a protease-resistant laminin-binding alpha6 integrin.

45. The isolated polypeptide of claim 44, wherein the alpha6 integrin is resistant to urokinase.

46. A method of inhibiting cleavage of a laminin-binding alpha6 integrin in a sample, the method comprising treating the sample with a urokinase inhibitor.

47. The method of claim 46, wherein the urokinase inhibitor selected from the group consisting of: a urokinase ATF, amiloride, PAI-1, PAI-2, B428, B623, p-aminobenzamidine, epigallo-cathecin-3 gallate, and alpha-N-benzylsulfonyl-p-aminophenylalanine.

48. A method of treating a subject, the method comprising:
identifying a subject in need of an agent that inhibits cleavage of a laminin-binding alpha6 integrin, and
administering to the subject an agent that inhibits alpha6 cleavage, wherein the agent interacts with uPAR, a laminin-binding alpha6 integrin, a tetraspanin, or urokinase, thereby treating the subject.

49. The method of claim 48, wherein the agent is a urokinase inhibitor.

50. The method of claim 49, wherein the urokinase inhibitor is one or more of the group consisting of: a urokinase ATF, amiloride, PAI-1, PAI-2, B428, B623, p-aminobenzamidine, epigallo-cathecin-3 gallate, and alpha-N-benzylsulfonyl-p-aminophenylalanine.

51. The method of claim 48, wherein the agent inhibits interaction of uPAR with alpha6.

52. The method of claim 48, wherein the subject has a proliferative disorder or is at risk for a proliferative disorder.

53. The method of claim 48, wherein the agent inhibits interaction of the laminin-binding alpha6 integrin with uPAR.

54. The method of claim 48, wherein the agent interacts with a tetraspanin.

55. The method of claim 54, wherein the tetraspanin is CD151.

56. A method of treating a subject, the method comprising:
identifying a subject in need of an activator of alpha6p activity or of inactivation of alpha6 activity, and
administering to the subject an agent that increases alpha6 integrin cleavage,
thereby treating the subject.

57. The method of claim 56, wherein the agent is urokinase or an activator of urokinase.

58. The method of claim 56, wherein the subject has a thrombotic disorder.

59. the method of claim 56, wherein the subject has an embolism.

60. The method of claim 56, wherein the agent is a growth factor or hormone.

61. An isolated protein ligand that binds to alpha6-interacting protein and inhibits interaction alpha6 integrin and the alpha6 interacting protein

62. The ligand of claim 61 wherein the protein is selected from the group consisting of: uPAR, urokinase, and a tetraspanin.

63. A method of providing a truncated integrin, the method comprising:
providing a cell that produces alpha6 integrin; and
contacting the cell with urokinase under conditions in which the alpha6 is cleaved.

64. The method of claim 63 wherein the cell contains a heterologous gene that encodes the alpha6 integrin.
